(19) United States
(12) Patent Application Publication
(10) Pub. No.: US 2023/0257431 A1
(43) Pub. Date: Aug. 17, 2023

(54) CSRP3 (CYSTEINE AND GLYCINE RICH PROTEIN 3) GENE THERAPY

(71) Applicant: Spacecraft Seven, LLC, Cranbury, NJ (US)

(72) Inventors: Christopher Dean HERZOG, Cranbury, NJ (US); Chester Bittencort SACRAMENTO, Cranbury, NJ (US); Raj PRABHAKAR, Cranbury, NJ (US); David RICKS, Cranbury, NJ (US)

(21) Appl. No.: 18/019,396

(22) PCT Filed: Aug. 3, 2021
(86) PCT No.: PCT/US2021/044412

§ 371 Date: Feb. 2, 2023

Related U.S. Application Data
(60) Provisional application No. 63/061,727, filed on Aug. 5, 2020.

(51) Int. Cl. C07K 14/47; C12N 15/86; A61P 9/04

(52) U.S. Cl. CPC C07K 14/4705; A61P 9/04; C07K 14/4716; C12N 15/86; C12N 2750/14143

(57) ABSTRACT

Provided herein is a gene therapy for CSRP3 (Cysteine and Glycine Rich Protein 3)-related gene deficits associated with cardiomyopathy, e.g. using an adeno-associated virus (AAV) vector. The promoter of the vector may be a MHCK7 promoter or a cardiac troponin T (HTNNT2) promoter. The capsid may be an AAV9 or AAVrh74 capsid or a functional variant thereof. Other promoters or capsids may be used. Further provided are methods of treatment, such as by intravenous, intracoronary, intracarotid or intracardiac administration of the rAAV vector, and other compositions and methods.

### CROSS-REFERENCE TO RELATED APPLICATIONS

This application claims priority to U.S. Application No. 63/061,727, filed on Aug. 5, 2020, the contents of which are incorporated by reference herein in their entireties.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ROPA_020_01WO_ST25.txt. The text file is about 120 KB, created on Aug. 3, 2021, and is being submitted electronically via EFS-Web.

### BACKGROUND

Cysteine and glycine rich protein 3 (CSRP3) encodes Muscle LIM Protein (MLP). Genetic defects in CSRP3 are associated with autosomal dominant cardiomyopathy, both hypertrophic cardiomyopathy (HCM) and dilated cardiomyopathy (DCM), as Autosomal dominant mutations in different domains of the protein are linked with different phenotypes. Loss-of-function mutations that decrease MLP levels can cause protein mislocalization and proteasome-mediated degradation, resulting in disruption of normal signaling pathways in cardiac and skeletal muscle. Changes in MLP levels or intracellular localization are also associated with skeletal myopathies, including facioscapulohumeral muscular dystrophy, nemaline myopathy, and limb girdle muscular dystrophy type 2B. Changes in levels of the isoform MLP-b protein or disregulation of the MLP:MLP-b ratio have been detected in limb girdle muscular dystrophy type 2A, Duchenne muscular dystrophy, and dermatomyositis patients.

CSRP3 patients exhibit variable symptoms depending on the specific mutation, but general symptoms include obstructive HCM or DCM, ventricular hypertrophy (with interventricular septum in the range of 14-32 mm), ventricular tachycardia, exercise intolerance, angina. Mild NYHA (New York Heart Association) scores of I-II are common. Sudden cardiac death has been observed, for example in a family carrying the C58G mutation. In one study, the majority of C58G carriers who provided muscle biopsies complained of exertional myalgias and cramps at presentation.

There is an unmet need for therapy for CSRP3-related diseases or disorders. The gene therapies provided herein address this need.

### SUMMARY

The present invention relates generally to gene therapy for a disease or disorder, e.g., a cardiac disease or disorder, using a vector expressing MLP or a functional variant thereof.

In one aspect, the disclosure provides polynucleotide, comprising an expression cassette and optionally flanking adeno-associated virus (AAV) inverted terminal repeats (ITRs), wherein the polynucleotide comprises a polynucleotide sequence encoding Muscle LIM Protein (MLP) or a functional variant thereof, operatively linked to a promoter.

In some embodiments, the promoter is a cardiac-specific promoter.

In some embodiments, the promoter is a muscle-specific promoter.

In some embodiments, the promoter is a cardiomyocyte-specific promoter.

In some embodiments, the promoter is a MHCK7 promoter.

In some embodiments,the MHCK7 promoter shares at least 75%, 80%, 85%, 90%, 95%, 96%, 97%, 98%, 99% or 100% identity with SEQ ID NO: 31.

In some embodiments, the promoter is a cardiac troponin T (hTNNT2) promoter.

In some embodiments, the hTNNT2 promoter shares at least 75%, 80%, 85%, 90%, 95%, 96%, 97%, 98%, 99% or 100% identity with SEQ ID NO: 32.

In some embodiments, the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

In some embodiments, the promoter is a ubiquitous promoter, optionally a CMV promoter or a CAG promoter.

In some embodiments, the expression cassette comprises a polyA signal.

In some embodiments, the polyA signal is a human growth hormone (hGH) polyA.

In some embodiments, the expression cassette comprises a Woodchuck Hepatitis Virus Posttranscriptional Regulatory Element (WPRE), optionally a WPRE(x).

In some embodiments, the Muscle LIM Protein (MLP) or a functional variant thereof is an MLP.

In some embodiments, the MLP is a human MLP.

In some embodiments, the MLP is MLP isoform A.

In some embodiments, the MLP shares at least 75%, 80%, 85%, 90%, 95%, 96%, 97%, 98%, 99% or 100% identity with SEQ ID NO: 1.

In some embodiments, the MLP is MLP isoform B.

In some embodiments, the MLP shares at least 75%, 80%, 85%, 90%, 95%, 96%, 97%, 98%, 99% or 100% identity with SEQ ID NO: 2.

In some embodiments, the MLP shares at least 75%, 80%, 85%, 90%, 95%, 96%, 97%, 98%, 99% or 100% identity with SEQ ID NO: 3.

In some embodiments, the MLP shares at least 75%, 80%, 85%, 90%, 95%, 96%, 97%, 98%, 99% or 100% identity with SEQ ID NO: 4.

In some embodiments, the polynucleotide sequence encoding MLP is a Cysteine And Glycine Rich Protein 3 (CSRP3) polynucleotide.

In some embodiments, the CSRP3 polynucleotide is a human CSRP3 polynucleotide.

In some embodiments, the polynucleotide sequence encoding MLP shares at least 75%, 80%, 85%, 90%, 95%, 96%, 97%, 98%, 99% or 100% identity with SEQ ID NO: 5.

In some embodiments, the polynucleotide sequence encoding MLP shares at least 75%, 80%, 85%, 90%, 95%, 96%, 97%, 98%, 99% or 100% identity with SEQ ID NO: 7.

In some embodiments, the polynucleotide comprises at least about 2.4 kb, at most about 2.6 kb, or between about 2.4 kb and about 2.6 kb.

In some embodiments, the polynucleotide comprises at least about 3.0 kb, at most about 3.3 kb, or between about 3.0 kb and about 3.3 kb.

In some embodiments, the polynucleotide comprises at least about 2.4 kb, least about 2.6 kb, least about 3.0 kb, at least about 3.3 kb, at least about 3.5 kb, at least about 3.7 kb, at least about 3.9 kb, at least about 4.1 kb., or at least about 4.3 kb.

In some embodiments, the polynucleotide comprises least about 2.6 kb, least about 3.0 kb, at most about 3.3 kb, at most about 3.5 kb, at most about 3.7 kb, at most about 3.9 kb, at most about 4.1 kb., at most about 4.3 kb, or at most about 4.5 kb.

In some embodiments, the expression cassette shares at least 75%, 80%, 85%, 90%, 95%, 96%, 97%, 98%, 99% or 100% identity with any one of SEQ ID NOs: 8-11.

In some embodiments, the polynucleotide shares at least 75%, 80%, 85%, 90%, 95%, 96%, 97%, 98%, 99% or 100% identity with any one of SEQ ID NOs: 12-15.

In some embodiments, the expression cassette is flanked by 5′ and 3′ inverted terminal repeats (ITRs), optionally AAV2 ITRs, optionally ITRs that shares at least 75%, 80%, 85%, 90%, 95%, 96%, 97%, 98%, 99% or 100% identity with any one of SEQ ID NO: 20-26.

In some embodiments, the polynucleotide is self-complementary.

In some embodiments, the polynucleotide comprises the expression cassette and a reverse complement of the expression cassette.

In some embodiments, the expression cassette and the reverse complement of the expression cassette are flanked by 5′ and 3′ inverted terminal repeats (ITRs), optionally AAV2 ITRs, optionally an ITR that shares at least 75%, 80%, 85%, 90%, 95%, 96%, 97%, 98%, 99% or 100% identity with SEQ ID NO: 23 or SEQ ID NO: 26.

In another aspect, the disclosure provides a gene therapy vector, comprising a polynucleotide of the disclosure.

In some embodiments, the gene therapy vector is a recombinant adeno-associated virus (rAAV) vector.

In some embodiments, the rAAV vector is an AAV9 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 77.

In some embodiments, the rAAV vector is an AAVrh10 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 79.

In some embodiments, the rAAV vector is an AAV6 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 78.

In some embodiments, the rAAV vector is an AAVrh74 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 80.

In some embodiments, the rAAV vector is a self-complementary AAV vector.

In another aspect, the disclosure provides a method of treating and/or preventing a disease or disorder in a subject in need thereof, comprising administering a vector of the disclosure to the subject.

In some embodiments, the disease or disorder is a cardiac disorder.

In some embodiments, the disease or disorder is heart failure.

In some embodiments, the disease or disorder is hypertrophic cardiomyopathy.

In some embodiments, the disease or disorder is dilated cardiomyopathy.

In some embodiments, the subject is a mammal.

In some embodiments, the subject is a primate.

In some embodiments, the subject is a human.

In some embodiments, the subject has a mutation in the CSRP3 gene that causes an amino acid substitution selected from C58G, L44P, S54R, E55G, and/or K69R, relative to a human CSRP3 encoding a human MLP having the sequence of SEQ ID NO: 1.

In some embodiments, the vector is administered by intravenous injection, intracardiac injection, intracardiac infusion, and/or cardiac catheterization.

In some embodiments, the administration increases MLP expression by at least about 5%.

In some embodiments, the administration increases MLP expression by at least about 30%.

In some embodiments, the administration increases MLP expression by at least about 70%.

In some embodiments, the administration increases MLP expression by about 5% to about 10%.

In some embodiments, the administration increases MLP expression by about 30% to about 50%.

In some embodiments, the administration increases MLP expression by about 70% to about 100%.

In some embodiments, the method treats and/or prevents the disease or disorder.

In another aspect, the disclosure provides a pharmaceutical composition comprising a vector of the disclosure.

In another aspect, the disclosure provides a kit comprising a vector or pharmaceutical composition of the disclosure, and optionally instructions for use.

In another aspect, the disclosure provides a use of a composition of the disclosure in treating a disease or disorder, optionally according to any of the methods disclosed herein.

In another aspect, the disclosure provides a composition of the disclosure for use in treating a disease or disorder, optionally according to any of the methods disclosed herein.

In another aspect, the disclosure provides a method of expressing Muscle LIM Protein (MLP) or a functional variant thereof, comprising contacting a cell with a vector of the disclosure.

In some embodiments, the cell is a cardiomyocyte.

In some embodiments, the cardiomyocyte is a human cardiomyocyte.

In some embodiments, the promoter is an MHCK7 promoter and wherein the expression level of the MLP is at least 2-fold greater than the expression level of MLP in a cell transduced with a vector having an hTNNT2 promoter.

In some embodiments, the promoter is an MHCK7 promoter and wherein the expression level of the MLP is between 2-fold greater and 10-fold greater than the expression level of MLP in a cell transduced with a vector having an hTNNT2 promoter.

Various other aspects and embodiments are disclosed in the detailed description that follows. The invention is limited solely by the appended claims.

### BRIEF DESCRIPTION OF FIGURES

FIG. 1 shows a vector diagram of a non-limiting example of a vector genome. The full polynucleotide sequence of the vector genome is SEQ ID NO: 12. The capitalized portion is the expression cassette (SEQ ID NO: 8).

FIG. 2 shows a vector diagram of a non-limiting example of a vector genome. The full polynucleotide sequence of the vector genome is SEQ ID NO: 13. The capitalized portion is the expression cassette (SEQ ID NO: 9).

FIG. 3 shows a vector diagram of a non-limiting example of a vector genome. The full polynucleotide sequence of the vector genome is SEQ ID NO: 14. The capitalized portion is the expression cassette (SEQ ID NO: 10).

FIG. 4 shows a vector diagram of a non-limiting example of a vector genome. The full polynucleotide sequence of the vector genome is SEQ ID NO: 15. The capitalized portion is the expression cassette (SEQ ID NO: 11).

FIG. 5A shows CSRP3 expression in transduced CHO-Lec2.

FIG. 5B shows CSRP3 expression in transduced cardiomyocytes (differentiated AC16 cell line - Sigma-Aldrich® cat# SCC109). The cells were transduced with 3E5 MOI from each vector; after 6 days the cells lysates were collected, and a Western Blot performed using an anti-CSRP3 Polyclonal antibody (Thermo-Fisher® PA5-29155 1:1000).

### DETAILED DESCRIPTION OF THE INVENTION

The present disclosure provided gene therapy vectors for CSPRP3 that delivery a polynucleotide encoding MLP, along with method of use, and other compositions and methods. Treatment of CSPRP3-related disorder is complicated by autosomal dominant nature of most forms of CSPRP3-related disorders and evidence suggesting that the level of protein expression and balance between MLP isoforms is crucial to normal function in healthy subjects. Moreover, successful gene therapy in the heart is unpredictable. Cardiomyocytes are a particularly challenging cell type to target with gene therapy. The compositions and methods disclosed herein address this problem.

### Definitions

The section headings are for organizational purposes only and are not to be construed as limiting the subject matter described to particular aspects or embodiments.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus a query sequence “shares at least x% identity to” a subject sequence if, when the query sequence is aligned to the subject sequence, at least x% (rounded down) of the residues in the subject sequence are aligned as an exact match to a corresponding residue in the query sequence. Where the subject sequence has variable positions (e.g., residues denoted X), an alignment to any residue in the query sequence is counted as a match.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operable linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in an CSRP3 gene or deletion of all or a part of CSRP3 gene, or of gene regulatory sequences, that causes aberrant expression of the MLP protein. “Subject” and “patient” are used interchangeably herein. The subject treated by the methods described herein may be an adult or a child. Subjects may range in age.

As used herein, the term “variant” or “functional variant” refer, interchangeably, to a protein that has one or more amino-acid substitutions, insertions, or deletion compared to a parental protein that retains one or more desired activities of the parental protein.

As used herein, “genetic disruption” refers to a partial or complete loss of function or aberrant activity in a gene. For example, a subject may suffer from a genetic disruption in expression or function in the CSRP3 gene that decreases expression or results in loss or aberrant function of the MLP protein in at least some cells (e.g., cardiac cells) of the subject.

As used herein, “treating” refers to ameliorating one or more symptoms of a disease or disorder. The term “preventing” refers to delaying or interrupting the onset of one or more symptoms of a disease or disorder or slowing the progression of CSRP3-related disease or disorder, e.g., hypertrophic cardiomyopathy (HCM), dilated cardiomyopathy (DCM), or skeletal myopathy.

### MLP Protein or Polyncleotide

The present disclosure contemplates compositions and methods of use related to Muscle LIM Protein (MLP) protein. Various mutations in CSRP3 are known to be associated with hypertrophic cardiomyopathy (HCM) or dilated cardiomyopathy (DCM). Both inherited and de novo mutations have been observed. In some cases, a heterozygous missense mutation is sufficient to cause disease.

The polypeptide sequence of MLP is as follows:

MPNWGGGAKCGACEKTVYHAEEIQCNGRSFHKTCFHCMACRKALDSTTVA
AHESEIYCKVCYGRRYGPKGIGYGQGAGCLSTDTGEHLGLQFQQSPKPAR
SVTTSNPSKFTAKFGESEKCPRCGKSVYAAEKVMGGGKPWHKTCFRCAIC
GKSLESTNVTDKDGELYCKVCYAKNFGPTGIGFGGLTQQVEKKE(SEQ I
D NO: 1).

A second isoform of MLP has the following polypeptide sequence:

MPNWGGGAKCGACEKTVYHAEEIQCNGRSFHKTCFHCSPQSRHAQLPPAT
LPNSLRSLESPRSALDVASQSMLLRRLWEVASLGTRPVSAVPSVGRVWSP
QMSLTKMGNFIAKFAMPKILAPRVLGLEALHNKWKRKNEEVRRFSDFLRA
(SEQ ID NO: 2).

Another isoform of MLP has the following polypeptide sequence:

MPNWGGGAKCGACEKTVYHAEEIQCNGRSFHKTCFHCLC(SEQ ID NO:
3).

Another isoform of MLP has the following polypeptide sequence:

MPNWGGGAKCGACEKTVYHAEEIQCNGRSFHKTCFHCTLAQDLFPLCHLW
EESGVHKC(SEQ ID NO: 4).

In some embodiments, the MLP protein comprises a polypeptide sequence at least 75%, 80%, 85%, 90%, 92%, 93%, 94%, 95%, 96%, 97%, 98%, 99%, or 100% identical to any one of SEQ ID NOs: 1-4.

In some embodiments, the disclosure provides a recombinant adeno-associated virus (rAAV) virion, comprising a capsid and a vector genome, wherein the vector genome comprises a polynucleotide sequence encoding an MLP or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an MLP, operatively linked to a promoter. The polynucleotide encoding the MLP may comprise a polynucleotide sequence at least 75%, 80%, 85%, 90%, 92%, 93%, 94%, 95%, 96%, 97%, 98%, 99%, or 100% identical to:

ATGCCAAACTGGGGCGGAGGCGCAAAATGTGGAGCCTGTGAAAAGACCGT
CTACCATGCAGAAGAAATCCAGTGCAATGGAAGGAGTTTCCACAAGACGT
GTTTCCACTGCATGGCCTGCAGGAAGGCTCTTGACAGCACGACAGTCGCG
GCTCATGAGTCGGAGATCTACTGCAAGGTGTGCTATGGGCGCAGATATGG
CCCCAAAGGGATCGGGTATGGACAAGGCGCTGGCTGTCTCAGCACAGACA
CGGGCGAGCATCTCGGCCTGCAGTTCCAACAGTCCCCAAAGCCGGCACGC
TCAGTTACCACCAGCAACCCTTCCAAATTCACTGCGAAGTTTGGAGAGTC
CGAGAAGTGCCCTCGATGTGGCAAGTCAGTCTATGCTGCTGAGAAGGTTA
TGGGAGGTGGCAAGCCTTGGCACAAGACCTGTTTCCGCTGTGCCATCTGT
GGGAAGAGTCTGGAGTCCACAAATGTCACTGACAAAGATGGGGAACTTTA
TTGCAAAGTTTGCTATGCCAAAAATTTTGGCCCCACGGGTATTGGGTTTG
GAGGCCTTACACAACAAGTGGAAAAGAAAGAA(SEQ ID NO: 5).

Optionally, the polynucleotide sequence encoding the vector genome may comprise a Kozak sequence, including but not limited to GCCACCATGG (SEQ ID NO: 6). Kozak sequence may overlap the polynucleotide sequence encoding an MLP protein or a functional variant thereof. For example, the vector genome may comprise a polynucleotide sequence (with Kozak underlined) at least 75%, 80%, 85%, 90%, 92%, 93%, 94%, 95%, 96%, 97%, 98%, 99%, or 100% identical to:

gccaccATGCCAAACTGGGGCGGAGGCGCAAAATGTGGAGCCTGTGAAAA
GACCGTCTACCATGCAGAAGAAATCCAGTGCAATGGAAGGAGTTTCCACA
AGACGTGTTTCCACTGCATGGCCTGCAGGAAGGCTCTTGACAGCACGACA
GTCGCGGCTCATGAGTCGGAGATCTACTGCAAGGTGTGCTATGGGCGCAG
ATATGGCCCCAAAGGGATCGGGTATGGACAAGGCGCTGGCTGTCTCAGCA
CAGACACGGGCGAGCATCTCGGCCTGCAGTTCCAACAGTCCCCAAAGCCG
GCACGCTCAGTTACCACCAGCAACCCTTCCAAATTCACTGCGAAGTTTGG
AGAGTCCGAGAAGTGCCCTCGATGTGGCAAGTCAGTCTATGCTGCTGAGA
AGGTTATGGGAGGTGGCAAGCCTTGGCACAAGACCTGTTTCCGCTGTGCC
ATCTGTGGGAAGAGTCTGGAGTCCACAAATGTCACTGACAAAGATGGGGA
ACTTTATTGCAAAGTTTGCTATGCCAAAAATTTTGGCCCCACGGGTATTG
GGTTTGGAGGCCTTACACAACAAGTGGAAAAGAAAGAA(SEQ ID NO:
7).

In some embodiments, the Kozak sequence is an alternative Kozak sequence comprising or consisting of any one of:

(gcc)gccRccAUGG (SEQ ID NO: 16);

(gcc)gccRccAUGC (SEQ ID NO: 17);

AGNNAUGN;

ANNAUGG;

ANNAUGC;

ACCAUGG;

ACCAUGC;

GACACCAUGG (SEQ ID NO: 18);

and

GACACCAUGC (SEQ ID NO: 19).

In some embodiments, the vector genome comprises no Kozak sequence.

### Vector Genome

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be from the same serotype as the capsid or a different serotype (e.g., AAV2 ITRs may be used).

In some embodiments, the 5′ ITR comprises a polynucleotide sequence at least 75%, 80%, 85%, 90%, 92%, 93%, 94%, 95%, 96%, 97%, 98%, 99%, or 100% identical to:

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT(SEQ ID N
O: 20)

In some embodiments, the 5′ ITR comprises a polynucleotide sequence at least 75%, 80%, 85%, 90%, 92%, 93%, 94%, 95%, 96%, 97%, 98%, 99%, or 100% identical to:

GCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGA
GTGGCCAACTCCATCACTAGGGGTTCCTTGTAGTTAATGATTAACCCGCC
ATGCTACTTATCTACGTA(SEQ ID NO: 21)

In some embodiments, the 5′ ITR comprises a polynucleotide sequence at least 75%, 80%, 85%, 90%, 92%, 93%, 94%, 95%, 96%, 97%, 98%, 99%, or 100% identical to:

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTA(SEQ ID NO: 22)

In some embodiments, the 5′ ITR comprises a polynucleotide sequence at least 75%, 80%, 85%, 90%, 92%, 93%, 94%, 95%, 96%, 97%, 98%, 99%, or 100% identical to:

TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT(SEQ
ID NO: 23)

In some embodiments, the 3′ ITR comprises a polynucleotide sequence at least 75%, 80%, 85%, 90%, 92%, 93%, 94%, 95%, 96%, 97%, 98%, 99%, or 100% identical to:

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(SEQ ID N
O: 24)

In some embodiments, the 3′ ITR comprises a polynucleotide sequence at least 75%, 80%, 85%, 90%, 92%, 93%, 94%, 95%, 96%, 97%, 98%, 99%, or 100% identical to:

TACGTAGATAAGTAGCATGGCGGGTTAATCATTAACTACAAGGAACCCCT
AGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCA
GTGAGCGAGCGAGCGCGC(SEQ ID NO: 25)

In some embodiments, the 3′ ITR comprises a polynucleotide sequence at least 75%, 80%, 85%, 90%, 92%, 93%, 94%, 95%, 96%, 97%, 98%, 99%, or 100% identical to:

AGGAACCCCTAGTGATGGAGACTCCCTCTCTGCGCGCTCGCTCGCTCACT
GAGGCCGGGCGACCAAAGGTCGCCCGACGCCCGGGCTTTGCCCGGGCGGC
CTCAGTGAGCGAGCGAGCGCGCAGAGAGGGAGT(SEQ ID NO: 26)

In some embodiments the vector genome comprises one or more filler sequences, e.g., at least 75%, 80%, 85%, 90%, 92%, 93%, 94%, 95%, 96%, 97%, 98%, 99%, or 100% identical to:

GCGGCAATTCAGTCGATAACTATAACGGTCCTAAGGTAGCGATTTAAATA
CGCGCTCTCTTAAGGTAGCCCCGGGACGCGTCAATTGACTACAAACCGAG
TATCTGCAGAGGGCCCTGCGTATG (SEQ ID NO: 27);

CTTCTGAGGCGGAAAGAACCAGATCCTCTCTTAAGGTAGCATCGAGATTT
AAATTAGGGATAACAGGGTAATGGCGCGGGCCGC (SEQ ID NO: 28)
;

or

GTTACCCAGGCTGGAGTGCAGTGGCACATTTCTGCTCACTGCAACCTCCT
CCTCCCTGGGTTC (SEQ ID NO: 29).

### Promoters

In some embodiments, the polynucleotide sequence encoding an MLP protein or functional variant thereof is operably linked to a promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least 75%, 80%, 85%, 90%, 92%, 93%, 94%, 95%, 96%, 97%, 98%, 99%, or 100% identical to:

ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCATGGTCGA
GGTGAGCCCCACGTTCTGCTTCACTCTCCCCATCTCCCCCCCCTCCCCAC
CCCCAATTTTGTATTTATTTATTTTTTAATTATTTTGTGCAGCGATGGGG
GCGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCG
CTCCGAAAGTTTCCTTTTATGGCGAGGCGGCGGCGGCGGCGGCCCTATAA
AAAGCGAAGCGCGCGGCGGGCGG(SEQ ID NO: 30)

In some embodiments, a polynucleotide sequence encoding an MLP protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some cases, the promoter is a ubiquitous promoter. A “ubiquitous promoter” refers to a promoter that is not tissue-specific under experimental or clinical conditions. In some cases, the ubiquitous promoter is any one of CMV, CAG, UBC, PGK, EF1-alpha, GAPDH, SV40, HBV, chicken beta-actin, and human beta-actin promoters.

In some embodiments, the promoter sequence is selected from Table 3. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOS 31-51.

TABLE 3

| | | |
|---|---|---|
| PROMOTER | SEQUENCE | SEQ ID NO: |
| MHCK7 | ACCCTTCAGATTAAAAATAACTGAGGTAAGGGCCTGGGTAG GGGAGGTGGTGTGAGACGCTCCTGTCTCTCCTCTATCTGCCC ATCGGCCCTTTGGGGAGGAGGAATGTGCCCAAGGACTAAAA AAAGGCCATGGAGCCAGAGGGGCGAGGGCAACAGACCTTTC ATGGGCAAACCTTGGGGCCCTGCTGTCTAGCATGCCCCACTA CGGGTCTAGGCTGCCCATGTAAGGAGGCAAGGCCTGGGGAC ACCCGAGATGCCTGGTTATAATTAACCCAGACATGTGGCTGC CCCCCCCCCCCCAACACCTGCTGCCTCTAAAAATAACCCTGT CCCTGGTGGATCCCCTGCATGCGAAGATCTTCGAACAAGGCT GTGGGGGACTGAGGGCAGGCTGTAACAGGCTTGGGGGCCAG GGCTTATACGTGCCTGGGACTCCCAAAGTATTACTGTTCCAT GTTCCCGGCGAAGGGCCAGCTGTCCCCCGCCAGCTAGACTCA GCACTTAGTTTAGGAACCAGTGAGCAAGTCAGCCCTTGGGGC AGCCCATACAAGGCCATGGGGCTGGGCAAGCTGCACGCCTG GGTCCGGGGTGGGCACGGTGCCCGGGCAACGAGCTGAAAGC TCATCTGCTCTCAGGGGCCCCTCCCTGGGGACAGCCCCTCCT GGCTAGTCACACCCTGTAGGCTCCTCTATATAACCCAGGGGC ACAGGGGCTGCCCTCATTCTACCACCACCTCCACAGCACAGA CAGACACTCAGGAGCCAGCCAG | 31 |
| Human cardiac troponin T | CTCAGTCCATTAGGAGCCAGTAGCCTGGAAGATGTCTTTACC CCCAGCATCAGTTCAAGTGGAGCAGCACATAACTCTTGCCCT | 33 |
| promoter (without exon 1) hTnnT2 / HTNNT2 | CTGCCTTCCAAGATTCTGGTGCTGAGACTTATGGAGTGTCTT GGAGGTTGCCTTCTGCCCCCCAACCCTGCTCCCAGCTGGCCC TCCCAGGCCTGGGTTGCTGGCCTCTGCTTTATCAGGATTCTCA AGAGGGACAGCTGGTTTATGTTGCATGACTGTTCCCTGCATA TCTGCTCTGGTTTTAAATAGCTTATCTGAGCAGCTGGAGGAC CACATGGGCTTATATGGCGTGGGGTACATGTTCCTGTAGCCT TGTCCCTGGCACCTGCCAAAATAGCAGCCAACACCCCCCACC CCCACCGCCATCCCCCTGCCCCACCCGTCCCCTGTCGCACAT TCCTCCCTCCGCAGGGCTGGCTCACCAGGCCCCAGCCCACAT GCCTGCTTAAAGCCCTCTCCATCCTCTGCCTCACCCAGT | |
| Human cardiac troponin T promoter (with exon 1, underlined) hTnnT2 / HTNNT2 | CTCAGTCCATTAGGAGCCAGTAGCCTGGAAGATGTCTTTACC CCCAGCATCAGTTCAAGTGGAGCAGCACATAACTCTTGCCCT CTGCCTTCCAAGATTCTGGTGCTGAGACTTATGGAGTGTCTT GGAGGTTGCCTTCTGCCCCCCAACCCTGCTCCCAGCTGGCCC TCCCAGGCCTGGGTTGCTGGCCTCTGCTTTATCAGGATTCTCA AGAGGGACAGCTGGTTTATGTTGCATGACTGTTCCCTGCATA TCTGCTCTGGTTTTAAATAGCTTATCTGAGCAGCTGGAGGAC CACATGGGCTTATATGGCGTGGGGTACATGTTCCTGTAGCCT TGTCCCTGGCACCTGCCAAAATAGCAGCCAACACCCCCCACC CCCACCGCCATCCCCCTGCCCCACCCGTCCCCTGTCGCACAT TCCTCCCTCCGCAGGGCTGGCTCACCAGGCCCCAGCCCACAT GCCTGCTTAAAGCCCTCTCCATCCTCTGCCTCACCCAGTCCCC GCTGAGACTGAGCAGACGCCTCCAGGATCTGTCGGCAG | 32 |
| Mouse a-cardiac myosin heavy chain promoter (αMHC) | GGTACCGGATCCTGCAAGGTCACACAAGGGTCTCCACCCACC AGGTGCCCTAGTCTCAATTTCAGTTTCCATGCCTTGTTCTCAC AATGCTGGCCTCCCCAGAGCTAATTTGGACTTTGTTTTTATTT CAAAAGGGCCTGAATGAGGAGTAGATCTTGTGCTACCCAGC TCTAAGGGTGCCCGTGAAGCCCTCAGACCTGGAGCCTTTGCA ACAGCCCTTTAGGTGGAAGCAGAATAAAGCAATTTTCCTTAA AGCCAAAATCCTGCCTCTAGACTCTTCTTCTCTGACCTCGGTC CCTGGGCTCTAGGGTGGGGAGGTGGGGCTTGGAAGAAGAAG GTGGGGAAGTGGCAAAAGCCGATCCCTAGGGCCCTGTGAAG | 34 |
| | TTCGGAGCCTTCCCTGTACAGCACTGGCTCATAGATCCTCCT CCAGCCAAACATAGCAAGAAGTGATACCTCCTTTGTGACTTC CCCAGGCCCAGTACCTGTCAGGTTGAAACAGGATTTAGAGA AGCCTCTGAACTCACCTGAACTCTGAAGCTCATCCACCAAGC AAGCACCTAGGTGCCACTGCTAGTTAGTATCCTACGCTGATA ATATGCAGAGCTGGGCCACAGAAGTCCTGGGGTGTAGGAAC TGACCAGTGACTTTTCAGTCGGCAAAGGTATGACCCCCTCAG CAGATGTAGTAATGTCCCCTTAGATCCCATCCCAGGCAGGTC TCTAAGAGGACATGGGATGAGAGATGTAGTCATGTGGCATT CCAAACACAGCTATCCACAGTGTCCCTTGCCCCTTCCACTTA GCCAGGAGGACAGTAACCTTAGCCTATCTTTCTTCCTCCCCA TCCTCCCAGGACACACCCCCTGGTCTGCAGTATTCATTTCTTC CTTCACGTCCCCTCTGTGACTTCCATTTGCAAGGCTTTTGACC TCTGCAGCTGCTGGAAGATAGAGTTTGGCCCTAGGTGTGGCA AGCCATCTCAAGAGAAAGCAGACAACAGGGGGACCAGATTT TGGAAGGATCAGGAACTAAATCACTGGCGGGCCTGGGGGTA GAAAAAAGAGTGAGTGAGTCCGCTCCAGCTAAGCCAAGCTA GTCCCCGAGATACTCTGCCACAGCTGGGCTGCTCGGGGTAGC TTTAGGAATGTGGGTCTGAAAGACAATGGGATTGGAAGACA TCTCTTTGAGTCTCCCCTCAACCCCACCTACAGACACACTCGT GTGTGGCCAGACTCCTGTTCAACAGCCCTCTGTGTTCTGACC ACTGAGCTAGGCAACCAGAGCATGGGCCCTGTGCTGAGGAT GAAGAGTTGGTTACCAATAGCAAAAACAGCAGGGGAGGGAG AACAGAGAACGAAATAAGGAAGGAAGAAGGAAAGGCCAGT CAATCAGATGCAGTCAGAAGAGATGGGAAGCCAACACACAG CTTGAGCAGAGGAAACAGAAAAGGGAGAGATTCTGGGCATA AGGAGGCCACAGAAAGAAGAGCCCAGGCCCCCCAAGTCTCC TCTTTATACCCTCATCCCGTCTCCCAATTAAGCCCACTCTTCT TCCTAGATCAGACCTGAGCTGCAGCGAAGAGACCCGTAGGG AGGATCACACTGGATGAAGGAGATGTGTGGAGAAGTCCAGG GAACCTAAGAGCCAGAGCCTAAAAGAGCAAGAGATAAAGGT GCTTCAAAGGTGGCCAGGCTGTGCACACAGAGGGTCGAGGA CTGGTGGTAGAGCCTCAAGATAAGGATGATGCTCAGAATGG GCGGGGGGGGGGATTCTGGGGGGGGGAGAGAGAAGGTGAG | |
| | AAGGAGCCTGGAACAGAGAATCTGGAAGCGCTGGAAACGAT ACCATAAAGGGAAGAACCCAGGCTACCTTTAGATGTAAATC ATGAAAGACAGGGAGAAGGGAAGCTGGAGAGAGTAGAAGG ACCCCGGGGCAAGACATTGAAGCAAGGACAAGCCAGGTTGA GCGCTCCGTGAAATCAGCCTGCTGAAGGCAGAGCCCTGGTAT GAGCACCAGAACAGCAGAGGCTAGGGTTAATGTCGAGACAG GGAACAGAAGGTAGACACAGGAACAGACAGAGACGGGGGA GCCAGGTAACAAAGGAATGGTCCTTCTCACCTGTGGCCAGA GCGTCCATCTGTGTCCACATACTCTAGAATGTTCATCAGACT GCAGGGCTGGCTTGGGAGGCAGCTGGAAAGAGTATGTGAGA GCCAGGGGAGACAAGGGGGCCTAGGAAAGGAAGAAGAGGG CAAACCAGGCCACACAAGAGGGCAGAGCCCAGAACTGAGTT AACTCCTTCCTTGTTGCATCTTCCATAGGAGGCAGTGGGAAC TCTGTGACCACCATCCCCCATGAGCCCCCACTACCCATACCA AGTTTGGCCTGAGTGGCATTCTAGGTTCCCTGAGGACAGAGC CTGGCCTTTGTCTCTTGGACCTGACCCAAGCTGACCCAATGT TCTCAGTACCTTATCATGCCCTCAAGAGCTTGAGAACCAGGC AGTGACATATTAGGCCATGGGCTAACCCTGGAGCTTGCACAC AGGAGCCTCAAGTGACCTCCAGGGACACAGCTGCAGACAGG TGGCCTTTATCCCCAAAGAGCAACCATTTGGCATAGGTGGCT GCAAATGGGAATGCAAGGTTGAATCAGGTCCCTTCAAGAAT ACTGCATGCAAGACCTAAGACCCCTGGAGAGAGGGGTATGC TCCTGCCCCCACCCACCATAAGGGGAGTGAACTATCCTAGGG GGCTGGCGACCTTGGGGAGACACCACATTACTGAGAGTGCT GAGCCCAGAAAAACTGACCGCCCTGTGTCCTGCCCACCTCCA CACTCTAGAGCTATATTGAGAGGTGACAGTAGATAGGGTGG GAGCTGGTAGCAGGGAGAGTGTTCCTGGGTGTGAGGGTGTA GGGGAAAGCCAGAGCAGGGGAGTCTGGCTTTGTCTCCTGAA CACAATGTCTACTTAGTTATAACAGGCATGACCTGCTAAAGA CCCAACATCTACGACCTCTGAAAAGACAGCAGCCCTGGAGG ACAGGGGTTGTCTCTGAGCCTTGGGTGCTTGATGGTGCCACA AAGGAGGGCATGAGTGTGAGTATAAGGCCCCAGGAGCGTTA GAGAAGGGCACTTGGGAAGGGGTCAGTCTGCAGAGCCCCTA TCCATGGAATCTGGAGCCTGGGGCCAACTGGTGTAAATCTCT | |
| | GGGCCTGCCAGGCATTCAAAGCAGCACCTGCATCCTCTGGCA GCCTGGGGAGGCGGAAGGGAGCAACCCCCCACTTATACCCT TTCTCCCTCAGCCCCAGGATTAACACCTCTGGCCTTCCCCCTT CCCACCTCCCATCAGGAGTGGAGGGTTGCAGAGGGAGGGTA AAAACCTACATGTCCAAACATCATGGTGCACGATATATGGAT CAGTATGTGTAGAGGCAAGAAAGGAAATCTGCAGGCTTAAC TGGGTTAATGTGTAAAGTCTGTGTGCATGTGTGTGTGTCTGA CTGAAAACGGGCATGGCTGTGCAGCTGTTCAGTTCTGTGCGT GAGGTTACCAGACTGCAGGTTTGTGTGTAAATTGCCCAAGGC AAAGTGGGTGAATCCCTTCCATGGTTTAAAGAGATTGGATGA TGGCCTGCATCTCAAGGACCATGGAAAATAGAATGGACACT CTATATGTGTCTCTAAGCTAAGGTAGCAAGGTCTTTGGAGGA CACCTGTCTAGAGATGTGGGCAACAGAGACTACAGACAGTA TCTGTACAGAGTAAGGAGAGAGAGGAGGGGGTGTAGAATTC TCTTACTATCAAAGGGAAACTGAGTCGTGCACCTGCAAAGTG GATGCTCTCCCTAGACATCATGACTTTGTCTCTGGGGAGCCA GCACTGTGGAACTTCAGGTCTGAGAGAGTAGGAGGCTCCCCT CAGCCTGAAGCTATGCAGATAGCCAGGGTTGAAAGGGGGAA GGGAGAGCCTGGGATGGGAGCTTGTGTGTTGGAGGCAGGGG ACAGATATTAAGCCTGGAAGAGAAGGTGACCCTTACCCAGT TGTTCAACTCACCCTTCAGATTAAAAATAACTGAGGTAAGGG CCTGGGTAGGGGAGGTGGTGTGAGACGCTCCTGTCTCTCCTC TATCTGCCCATCGGCCCTTTGGGGAGGAGGAATGTGCCCAAG GACTAAAAAAAGGCCATGGAGCCAGAGGGGCGAGGGCAAC AGACCTTTCATGGGCAAACCTTGGGGCCCTGCTGTCCTCCTG TCACCTCCAGAGCCAAGGGATCAAAGGAGGAGGAGCCAGGA CAGGAGGGAAGTGGGAGGGAGGGTCCCAGCAGAGGACTCC AAATTTAGGCAGCAGGCATATGGGATGGGATATAAAGGGGC TGGAGCACTGAGAGCTGTCAGAGATTTCTCCAACCCAGGTAA GAGGGAGTTTCGGGTGGGGGCTCTTCACCCACACCAGACCTC TCCCCACCTAGAAGGAAACTGCCTTTCCTGGAAGTGGGGTTC AGGCCGGTCAGAGATCTGACAGGGTGGCCTTCCACCAGCCT GGGAAGTTCTCAGTGGCAGGAGGTTTCCACAAGAAACACTG GATGCCCCTTCCCTTACGCTGTCTTCTCCATCTTCCTCCTGGG | |
| | GATGCTCCTCCCCGTCTTGGTTTATCTTGGCTCTTCGTCTTCA GCAAGATTTGCCCTGTGCTGTCCACTCCATCTTTCTCTACTGT CTCCGTGCCTTGCCTTGCCTTCTTGCGTGTCCTTCCTTTCCAC CCATTTCTCACTTCACCTTTTCTCCCCTTCTCATTTGTATTCAT CCTTCCTTCCTTCCTTCCTTCCTTCCTTCCTTCCTTCCTTCCTTC CTTTCTCCCTTCCTTCCTTCCTTCCTTCCTTCCTTCCTTCCTTCC TTCCTGTGTCAGAGTGCTGAGAATCACACCTGGGGTTCCCAC CCTTATGTAAACAATCTTCCAGTGAGCCACAGCTTCAGTGCT GCTGGGTGCTCTCTTACCTTCCTCACCCCCTGGCTTGTCCTGT TCCATCCTGGTCAGGATCTCTAGATTGGTCTCCCAGCCTCTGC TACTCCTCTTCCTGCCTGTTCCTCTCTCTGTCCAGCTGCGCCA CTGTGGTGCCTCGTTCCAGCTGTGGTCCACATTCTTCAGGATT CTCTGAAAAGTTAACCAGGTGAGAATGTTTCCCCTGTAGACA GCAGATCACGATTCTCCCGGAAGTCAGGCTTCCAGCCCTCTC TTTCTCTGCCCAGCTGCCCGGCACTCTTAGCAAACCTCAGGC ACCCTTACCCCACATAGACCTCTGACAGAGAAGCAGGCACTT TACATGGAGTCCTGGTGGGAGAGCCATAGGCTACGGTGTAA AAGAGGCAGGGAAGTGGTGGTGTAGGAAAGTCAGGACTTCA CATAGAAGCCTAGCCCACACCAGAAATGACAGACAGATCCC TCCTATCTCCCCCATAAGAGTTTGAGTCGACCCGCGGCCCCG AATTG | |
| Chicken cardiac troponin T promoter (cTnT) | GGGATAAAAGCAGTCTGGGCTTTCACATGACAGCATCTGGG GCTGCGGCAGAGGGTCGGGTCCGAAGCGCTGCCTTATCAGC GTCCCCAGCCCTGGGAGGTGACAGCTGGCTGGCTTGTGTCAG CCCCTCGGGCACTCACGTATCTCCGTCCGACGGGTTTAAAAT AGCAAAACTCTGAGGCCACACAATAGCTTGGGCTTATATGG GCTCCTGTGGGGGAAGGGGGAGCACGGAGGGGGCCGGGGCC GCTGCTGCCAAAATAGCAGCTCACAAGTGTTGCATTCCTCTC TGGGCGCCGGGCACATTCCTGCTGGCTCTGCCCGCCCCGGGG TGGGCGCCGGGGGGACCTTAAAGCCTCTGCCCCCCAAGGAG CCCTTCCCAGACAGCCGCCGGCACCCACCGCTCCGTGGGA | 35 |
| Human Creatine Kinase M (hCKM) | CTCTCAGCCCTGGAAGTCCTTGCTCACAGCCGAGGCGCCGAG AGCGCTTGCTCTGCCCAGATCTGCGCGAGTCTGGCGCCCGCG CTCTGAACGGCGTCGCTGCCCAGCCCCCTTCCCCGGGAGGTG GGAGCGGCCACCCAGGGCCCCGTGGCTGCCCTTGTAAGGAG GCGAGGCCCGAGGACACCCGAGACGCCCGGTTATAATTAAC CAGGACACGTGGCGAACCCCCCTCCAACACCTGCCCCCGAA CCCCCCCATACCCAGCGCCTCGGGTCTCGGCCTTTGCGGCAG AGGAGACAGCAAAGCGCCCTCTAAAAATAACTCCTTTCCCG GCGACCGAGACCCTCCCTGTCCCCCGCACAGCGGAAATCTCC CAGTGGCACCGAGGGGGCGAGGGTTAAGTGGGGGGGAGGGT GACCACCGCCTCCCACCCTTGCCCTGAGTTTGAATCTCTCCA ACTCAGCCAGCCTCAGTTTCCCCTCCACTCAGTCCCTAGGAG GAAGGGGCGCCCAAGCGCGGGTTTCTGGGGTTAGACTGCCC TCCATTGCAATTGGTCCTTCTCCCGGCCTCTGCTTCCTCCAGC TCACAGGGTATCTGCTCCTCCTGGAGCCACACCTTGGTTCCC CGAGGTGCCGCTGGGACTCGGGTAGGGGTGAGGGCCCAGGG GGCACAGGGGGAGCCGAGGGCCACAGGAAGGGCTGGTGGCT GAAGGAGACTCAGGGGCCAGGGGACGGTGGCTTCTACGTGC TTGGGACGTTCCCAGCCACCGTCCCATGTTCCCGGCGGGGGG CCAGCTGTCCCCACCGCCAGCCCAACTCAGCACTTGGTCAGG GTATCAGCTTGGTGGGGGGGCGTGAGCCCAGCCCCTGGGGC GGCTCAGCCCATACAAGGCCATGGGGCTGGGCGCAAAGCAT GCCTGGGTTCAGGGTGGGTATGGTGCGGGAGCAGGGAGGTG AGAGGCTCAGCTGCCCTCCAGAACTCCTCCCTGGGGACAACC CCTCCCAGCCAATAGCACAGCCTAGGTCCCCCTATATAAGGC CACGGCTGCTGGCCCTTCCTTTGGGTCAGTGTCACCTCCAGG ATACAGACA | 36 |
| Human beta-actin (HuBa) | GCCCAGCACCCCAAGGCGGCCAACGCCAAAACTCTCCCTCCT CCTCTTCCTCAATCTCGCTCTCGCTCTTTTTTTTTTTCGCAAAA GGAGGGGAGAGGGGGTAAAAAAATGCTGCACTGTGCGGCGA AGCCGGTGAGTGAGCGGCGCGGGGCCAATCAGCGTGCGCCG TTCCGAAAGTTGCCTTTTATGGCTCGAGCGGCCGCGGCGGCG | 37 |
| | CCCTATAAAACCCAGCGGCGCGACGCGCCACCACCGCCGAG TC | |
| Chicken beta-actin (CBA) | GGTCGAGGTGAGCCCCACGTTCTGCTTCACTCTCCCCATCTC CCCCCCCTCCCCACCCCCAATTTTGTATTTATTTATTTTTTAAT TATTTTGTGCAGCGATGGGGGCGGGGGGGGGGGGGGCGCGC GCCAGGCGGGGCGGGGCGGGGCGAGGGGCGGGGCGGGGCG AGGCGGAGAGGTGCGGCGGCAGCCAATCAGAGCGGCGCGCT CCGAAAGTTTCCTTTTATGGCGAGGCGGCGGCGGCGGCGGCC CTATAAAAAGCGAAGCGCGCGGCGGGCGGGA | 38 |
| Cytomegaloviru s (CMV) | TGGTGATGCGGTTTTGGCAGTACACCAATGGGCGTGGATAGC GGTTTGACTCACGGGGATTTCCAAGTCTCCACCCCATTGACG TCAATGGGAGTTTGTTTTGGCACCAAAATCAACGGGACTTTC CAAAATGTCGTAATAACCCCGCCCCGTTGACGCAAATGGGC GGTAGGCGTGTACGGTGGGAGGTCTATATAAGCAGAGCTCG TTTAGTGAACCG | 39 |
| Cytomegaloviru s (CMV) (second version) | TAGTTATTAATAGTAATCAATTACGGGGTCATTAGTTCATAG CCCATATATGGAGTTCCGCGTTACATAACTTACGGTAAATGG CCCGCCTGGCTGACCGCCCAACGACCCCCGCCCATTGACGTC AATAATGACGTATGTTCCCATAGTAACGCCAATAGGGACTTT CCATTGACGTCAATGGGTGGAGTATTTACGGTAAACTGCCCA CTTGGCAGTACATCAAGTGTATCATATGCCAAGTACGCCCCC TATTGACGTCAATGACGGTAAATGGCCCGCCTGGCATTATGC CCAGTACATGACCTTATGGGACTTTCCTACTTGGCAGTACAT CTACGTATTAGTCATCGCTATTACCATGGTGATGCGGTTTTG GCAGTACATCAATGGGCGTGGATAGCGGTTTGACTCACGGG GATTTCCAAGTCTCCACCCCATTGACGTCAATGGGAGTTTGT TTTGGCACCAAAATCAACGGGACTTTCCAAAATGTCGTAACA ACTCCGCCCCATTGACGCAAATGGGCGGTAGGCGTGTACGGT GGGAGGTCTATATAAGCAGAGCTGGTTTAGTGAACCGT | 40 |
| Cytomegaloviru s (CMV) (third version) | CGTTACATAACTTACGGTAAATGGCCCGCCTGGCTGACCGCC CAACGACCCCCGCCCATTGACGTCAATAATGACGTATGTTCC CATAGTAACGCCAATAGGGACTTTCCATTGACGTCAATGGGT GGAGTATTTACGGTAAACTGCCCACTTGGCAGTACATCAAGT GTATCATATGCCAAGTACGCCCCCTATTGACGTCAATGACGG TAAATGGCCCGCCTGGCATTATGCCCAGTACATGACCTTATG GGACTTTCCTACTTGGCAGTACATCTACGTATTAGTCATCGCT ATTACCATGGTGATGCGGTTTTGGCAGTACATCAATGGGCGT GGATAGCGGTTTGACTCACGGGGATTTCCAAGTCTCCACCCC ATTGACGTCAATGGGAGTTTGTTTTGGCACCAAAATCAACGG GACTTTCCAAAATGTCGTAACAACTCCGCCCCATTGACGCAA ATGGGCGGTAGGCGTGTACGGTGGGAGGTCTATATAAGCAG AGCT | 41 |
| CAG promoter (first version) | ACTTACGGTAAATGGCCCGCCTGGCTGACCGCCCAACGACCC CCGCCCATTGACGTCAATAATGACGTATGTTCCCATAGTAAC GCCAATAGGGACTTTCCATTGACGTCAATGGGTGGAGTATTT ACGGTAAACTGCCCACTTGGCAGTACATCAAGTGTATCATAT GCCAAGTACGCCCCCTATTGACGTCAATGACGGTAAATGGCC CGCCTGGCATTATGCCCAGTACATGACCTTATGGGACTTTCC TACTTGGCAGTACATCTACGTATTAGTCATCGCTATTACCAT GGTCGAGGTGAGCCCCACGTTCTGCTTCACTCTCCCCATCTC CCCCCCCTCCCCACCCCCAATTTTGTATTTATTTATTTTTTAAT TATTTTGTGCAGCGATGGGGGCGGGGGGGGGGGGGGCGCGC GCCAGGCGGGGCGGGGCGGGGCGAGGGGCGGGGCGGGGCG AGGCGGAGAGGTGCGGCGGCAGCCAATCAGAGCGGCGCGCT CCGAAAGTTTCCTTTTATGGCGAGGCGGCGGCGGCGGCGGCC CTATAAAAAGCGAAGCGCGCGGCGGGCGG | 42 |
| CAG promoter (second version) | CGTTACATAACTTACGGTAAATGGCCCGCCTGGCTGACCGCC CAACGACCCCCGCCCATTGACGTCAATAATGACGTATGTTCC CATAGTAACGCCAATAGGGACTTTCCATTGACGTCAATGGGT GGAGTATTTACGGTAAACTGCCCACTTGGCAGTACATCAAGT GTATCATATGCCAAGTACGCCCCCTATTGACGTCAATGACGG | 43 |
| | TAAATGGCCCGCCTGGCATTATGCCCAGTACATGACCTTATG GGACTTTCCTACTTGGCAGTACATCTACGTATTAGTCATCGCT ATTACCATGTCGAGGTGAGCCCCACGTTCTGCTTCACTCTCC CCATCTCCCCCCCCTCCCCACCCCCAATTTTGTATTTATTTAT TTTTTAATTATTTTGTGCAGCGATGGGGGCGGGGGGGGGGGG GGCGCGCGCCAGGCGGGGCGGGGCGGGGCGAGGGGCGGGG CGGGGCGAGGCGGAGAGGTGCGGCGGCAGCCAATCAGAGC GGCGCGCTCCGAAAGTTTCCTTTTATGGCGAGGCGGCGGCGG CGGCGGCCCTATAAAAAGCGAAGCGCGCGGCGGGCG | |
| Human EF1-alpha (EF1-α) | CAACCTTTGGAGCTAAGCCAGCAATGGTAGAGGGAAGATTC TGCACGTCCCTTCCAGGCGGCCTCCCCGTCACCACCCCCCCC AACCCGCCCCGACCGGAGCTGAGAGTAATTCATACAAAAGG ACTCGCCCCTGCCTTGGGGAATCCCAGGGACCGTCGTTAAAC TCCCACTAACGTAGAACCCAGAGATCGCTGCGTTCCCGCCCC CTCACCCGCCCGCTCTCGTCATCACTGAGGTGGAGAATAGCA TGCGTGAGGCTCCGGTGCCCGTCAGTGGGCAGAGCGCACAT CGCCCACAGTCCCCGAGAAGTTGGGGGGAGGGGTCGGCAAT TGAACGGGTGCCTAGAGAAGGTGGCGCGGGGTAAACTGGGA AAGTGATGTCGTGTACTGGCTCCGCCTTTTTCCCGAGGGTGG GGGAGAACCGTATATAAGTGCAGTAGTCGCCGTGAACGTT | 44 |
| Human Synapsin1 (Syn), short version | AGTGCAAGTGGGTTTTAGGACCAGGATGAGGCGGGGTGGGG GTGCCTACCTGACGACCGACCCCGACCCACTGGACAAGCAC CCAACCCCCATTCCCCAAATTGCGCATCCCCTATCAGAGAGG GGGAGGGGAAACAGGATGCGGCGAGGCGCGTGCGCACTGCC AGCTTCAGCACCGCGGACAGTGCCTTCGCCCCCGCCTGGCGG CGCGCGCCACCGCCGCCTCAGCACTGAAGGCGCGCTGACGT CACTCGCCGGTCCCCCGCAAACTCCCCTTCCCGGCCACCTTG GTCGCGTCCGCGCCGCCGCCGGCCCAGCCGGACCGCACCAC GCGAGGCGCGAGATAGGGGGGCACGGGCGCGACCATCTGCG CTGCGGCGCCGGCGACTCAGCGCTGCCTC | 45 |
| Human Synapsin1 (Syn) | AGTGCAAGTGGGTTTTAGGACCAGGATGAGGCGGGGTGGGG GTGCCTACCTGACGACCGACCCCGACCCACTGGACAAGCAC | 46 |
| with 3′ extension | CCAACCCCCATTCCCCAAATTGCGCATCCCCTATCAGAGAGG GGGAGGGGAAACAGGATGCGGCGAGGCGCGTGCGCACTGCC AGCTTCAGCACCGCGGACAGTGCCTTCGCCCCCGCCTGGCGG CGCGCGCCACCGCCGCCTCAGCACTGAAGGCGCGCTGACGT CACTCGCCGGTCCCCCGCAAACTCCCCTTCCCGGCCACCTTG GTCGCGTCCGCGCCGCCGCCGGCCCAGCCGGACCGCACCAC GCGAGGCGCGAGATAGGGGGGCACGGGCGCGACCATCTGCG CTGCGGCGCCGGCGACTCAGCGCTGCCTCAGTCTGCGGTGGG CAGCGGAGGAGTCGTGTCGTGCCTGAGAGCGCAG | |
| Human Synapsin1 (Syn) with 5′ extension | CTGCAGAGGGCCCTGCGTATGAGTGCAAGTGGGTTTTAGGAC CAGGATGAGGCGGGGTGGGGGTGCCTACCTGACGACCGACC CCGACCCACTGGACAAGCACCCAACCCCCATTCCCCAAATTG CGCATCCCCTATCAGAGAGGGGGAGGGGAAACAGGATGCGG CGAGGCGCGTGCGCACTGCCAGCTTCAGCACCGCGGACAGT GCCTTCGCCCCCGCCTGGCGGCGCGCGCCACCGCCGCCTCAG CACTGAAGGCGCGCTGACGTCACTCGCCGGTCCCCCGCAAAC TCCCCTTCCCGGCCACCTTGGTCGCGTCCGCGCCGCCGCCGG CCCAGCCGGACCGCACCACGCGAGGCGCGAGATAGGGGGGC ACGGGCGCGACCATCTGCGCTGCGGCGCCGGCGACTCAGCG CTGCCTC | 47 |
| Human CamKIIa (CaMKIIa) | ACTTGTGGACAAAGTTTGCTCTATTCCACCTCCTCCAGGCCCT CCTTGGGTCCATCACCCCAGGGGTGCTGGGTCCATCCCACCC CCAGGCCCACACAGGCTTGCAGTATTGTGTGCGGTATGGTCA GGGCGTCCGAGAGCAGGTTTCGCAGTGGAAGGCAGGCAGGT GTTGGGGAGGCAGTTACCGGGGCAACGGGAACAGGGCGTTT TGGAGGTGGTTGCCATGGGGACCTGGATGCTGACGAAGGCT CGCGAGGCTGTGAGCAGCCACAGTGCCCTGC | 48 |
| eSYN promoter | GACATTGATTATTGACTAGTTATTAATAGTAATCAATTACGG GGTCATTAGTTCATAGCCCATATATGGAGTTCCGCGTTACAT AACTTACGGTAAATGGCCCGCCTGGCTGACCGCCCAACGACC CCCGCCCATTGACGTCAATAATGACGTATGTTCCCATAGTAA CGCCAATAGGGACTTTCCATTGACGTCAATGGGTGGACTATT | 49 |
| | TACGGTAAACTGCCCACTTGGCAGTACATCAAGTGTATCATA TGCCAAGTACGCCCCCTATTGACGTCAATGACGGTAAATGGC CCGCCTGGCATTATGCCCAGTACATGACCTTATGGGACTTTC CTACTTGGCAGTACATCTACGTATTAGTCATCGCTATTACCAT GGCTGCAGAGGGCCCTGCGTATGAGTGCAAGTGGGTTTTAG GACCAGGATGAGGCGGGGTGGGGGTGCCTACCTGACGACCG ACCCCGACCCACTGGACAAGCACCCAACCCCCATTCCCCAAA TTGCGCATCCCCTATCAGAGAGGGGGAGGGGAAACAGGATG CGGCGAGGCGCGTCGCGACTGCCAGCTTCAGCACCGCGGAC AGTGCCTTCGCCCCCGCCTGGCGGCGCGCGCCACCGCCGCCT CAGCACTGAAGGCGCGCTGACGTCACTCGCCGGTCCCCCGCA AACTCCCCTTCCCGGCCACCTTGGTCGCGTCCGCGCCGCCGC CGGCCCAGCCGGACCGCACCACGCGAGGCGCGAGATAGGGG GGCACGGGCGCGACCATCTGCGCTGCGGCGCCGGCGACTCA GCGCTGCCTCAGTCTGCGGTGGGCAGCGGAGGAGTCGTGTC GTGCCTGAGAGCGCAGG | |

In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 31. In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 32. In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 33.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

### Other Regulatory Elements

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is:

ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCA(SEQ ID
NO: 50)

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 51-61.

TABLE 4

| 5′ UNTRANSLATED REGION | SEQUENCE | SEQ ID NO: |
|---|---|---|
| Human beta-actin exon/intron | CGCGTCCGCCCGCGAGCACAGAGCCTCGCCTTTGCCGATC CGCCGCCCGTCCACACCCGCCGCCAGGTAAGCCCGGCCAGCCGACCGGGGCATGCGGCCGCGGCCCTTCGCCCGTGCAGAGCCGCCGTCTGGGCCGCAGCGGGGGGCGCATGGGGCGGAACCGGACCGCCGTGGGGGGCGCGGGAGAAGCCCCTGGGCCTCCGGAGATGGGGGACACCCCACGCCAGTTCGCAGGCGCGAGGCCGCGCTCGGGCGGGCGCGCTCCGGGGGTGCCGCTCTCGGGGCGGGGGCAACCGGCGGGGTCTTTGTCTGAGCCGGGCTCTTGCCAATGGGGATCGCACGGTGGGCGCGGCGTAGCCCCCGTCAGGCCCGGTGGGGGCTGGGGCGCCATGCGCGTGCGCGCTGGTCCTTTGGGCGCTAACTGCGTGCGCGCTGGGAATTGGCGCTAATTGCGCGTGCGCGCTGGGACTCAATGGCGCTAATCGCGCGTGCGTTCTGGGGCCCGGGCGCTTGCGCCACTTCCTGCCCGAGCCGCTGGCGCCCGAGGGTGTGGCCGCTGCGTGCGCGCGCGCGACCCGGTCGCTGTTTGAACCGGGCGGAGGCGGGGCTGGCGCCCGGTTGGGAGGGGGTTGGGGCCTGGCTTCCTGCCGCGCGCCGCGGGGACGCCTCCGACCAGTGTTTGCCTTTTATGGTAATAACGCGGCCGGCCCGGCTTCCTTTGTCCCCAATCTGGGCGCGCGCCGGCGCCCCCTGGCGGCCTAAGGACTCGGCGCGCCGGAAGTGGCCAGGGCGGCAGCGGCTGCTCTTGGCGGCCCCGAGGTGACTATAGCCTTCTTTTGTGTCTTGATAGTTCGCCAGCCTCTGCTAACCATGTTCATGCCTTCTTCTTTTTCCTACAGCTCCTGGGCAACGTGCTGGTTATTGTGCTGTCTCATCATTTTGGCAAAGAATTC | 51 |
| Chicken beta-actin exon/intron + rabbit globin intron | GTCGCTGCGCGCTGCCTTCGCCCCGTGCCCCGCTCCGCG CCGCCTCGCGCCGCCCGCCCCGGCTCTGACTGACCGCGTT ACTCCCACAGGTGAGCGGGCGGGACGGCCCTTCTCCTCCG GGCTGTAATTAGCGCTTGGTTTAATGACGGCTTGTTTCTTTTCTGTGGCTGCGTGAAAGCCTTGAGGGGCTCCGGGAGGGCCCTTTGTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCGGCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTGAGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGCGGGGCCGCCTCGGGCCGGGGAGGGCTCGGGGGAGGGGCGCGGCGGCCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGCGGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCAGGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAGAATTC | 52 |
| Chimeric intron sequence | GGTAAGTTTAGTCTTTTTGTCTTTTATTTCAGGTCCCGGATCCGGTGGTGGTGCAAATCAAAGAACTGCTCCTCAGTGGATGTTGCCTTTACTTCTAGGCCTGTACGGAAGTGTTACTTCTGCTCTAAAAGCTGCGGAATTGTACCCGC | 53 |
| 5′UTR-Syn1 Hs | AGTCTGCGGTGGGCAGCGGAGGAGTCGTGTCGTGCCTGAGAGCGCAGCTGTGCTCCTGGGCACCGCGCAGTCCGCCCCCGCGGCTCCTGGCCAGACCACCCCTAGGACCCCCTGCCCCAAGTCGCA | 54 |
| CMV IE exon | TCAGATCGCCTGGAGAGGCCATCCACGCTGTTTTGACCTCCATAGTGGACACCGGGACCGATCCAGCCTCCGCGGCCGGGAACGGTGCATTGGAACGCGGATTCCCCGTGCCAAGAGTGAC | 55 |
| TPL-eMLP (adenovirus derived enhancer element) | CTCACTCTCTTCCGCATCGCTGTCTGCGAGGGCCAGCTGTT GGGCTCGCGGTTGAGGACAAACTCTTCGCGGTCTTTCCAG TACTCTTGGATCGGAAACCCGTCGGCCTCCGAACGGTACTCCGCCACCGAGGGACCTGAGCGAGTCCGCATCGACCGGATCGGAAAACCTCTCGAGAAAGGCGTCTAACCAGTCACAGTCGCAAGGTAGGCTGAGCACCGTGGCGGGCGGCAGCGGGTGGCGGTCGGGGTTGTTTCTGGCGGAGGTGCTGCTGATGATGTAATTAAAGTAGGCGGTCTTGAGACGGCGGATGGTCGAGGTGAGGTGTGGCAGGCTTGAGATCCAGCTGTTGGGGTGAGTACTCCCTCTCAAAAGCGGGCATTACTTCTGCGCTAAGATTGTCAGTTTCCAAAAACGAGGAGGATTTGATATTCACCTGGCCCGATCTGGCCATACACTTGAGTGACAATGACATCCACTTTGCCTTTCTCTCCACAGGTGTCCACTCCCAG | 56 |
| Human EF1-α intron/exon | CTTTTTCGCAACGGGTTTGCCGCCAGAACACAGGTAAGTG CCGTGTGTGGTTCCCGCGGGCCTGGCCTCTTTACGGGTTATGGCCCTTGCGTGCCTTGAATTACTTCCACCTGGCTCCAGTACGTGATTCTTGATCCCGAGCTGGAGCCAGGGGCGGGCCTTGCGCTTTAGGAGCCCCTTCGCCTCGTGCTTGAGTTGAGGCCTGGCCTGGGCGCTGGGGCCGCCGCGTGCGAATCTGGTGGCACCTTCGCGCCTGTCTCGCTGCTTTCGATAAGTCTCTAGCCATTTAAAATTTTTGATGACGTGCTGCGACGCTTTTTTTCTGGCAAGATAGTCTTGTAAATGCGGGCCAGGATCTGCACAC TGGTATTTCGGTTTTTGGGCCCGCGGCCGGCGACGGGGCC CGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTG | 57 |
| | TATCGCCCCGCCCTGGGCGGCAAGGCTGGCCCGGTCGGCACCAGTTGCGTGAGCGGAAAGATGGCCGCTTCCCGGCCCTGCTCCAGGGGGCTCAAAATGGAGGACGCGGCGCTCGGGAGAGCGGGCGGGTGAGTCACCCACACAAAGGAAAAGGGCCT TTCCGTCCTCAGCCGTCGCTTCATGTGACTCCACGGAGTAC CGGGCGCCGTCCAGGCACCTCGATTAGTTCTGGAGCTTTT GGAGTACGTCGTCTTTAGGTTGGGGGGAGGGGTTTTATGC GATGGAGTTTCCCCACACTGAGTGGGTGGAGACTGAAGTT AGGCCAGCTTGGCACTTGATGTAATTCTCCTTGGAATTTGGCCTTTTTGAGTTTGGATCTTGGTTCATTCTCAAGCCTCAGACAGTGGTTCAAAGTTTTTTTCTTCCATTTCAG | |
| Human EF1-α, intron A | GTAAGTGCCGTGTGTGGTTCCCGCGGGCCTGGCCTCTTTA CGGGTTATGGCCCTTGCGTGCCTTGAATTACTTCCACCTGG CTGCAGTACGTGATTCTTGATCCCGAGCTTCGGGTTGGAA GTGGGTGGGAGAGTTCGAGGCCTTGCGCTTAAGGAGCCCC TTCGCCTCGTGCTTGAGTTGAGGCCTGGCCTGGGCGCTGG GGCCGCCGCGTGCGAATCTGGTGGCACCTTCGCGCCTGTC TCGCTGCTTTCGATAAGTCTCTAGCCATTTAAAATTTTTGATGACCTGCTGCGACGCTTTTTTTCTGGCAAGATAGTCTTGTAAATGCGGGCCAAGATCTGCACACTGGTATTTCGGTTTTTGGGGCCGCGGGCGGCGACGGGGCCCGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTGTATCGCCCCGCCCTGGGCGGCAAGGCTGGCCCGGTCGGCACCAGTTGCGTGAGCGGAAAGATGGCCGCTTCCCGGCCCTGCTGCAGGGAGCTCAAAATGGAGGACGCGGCGCTCGGGAGAGCGGGCGGGTGAGTCACCCACACAAAGGAAAAGGGCCTTTCCGTCCTCAGCCGTCGCTTCATGTGACTCCACGGAGTACCGGGCGCCGTCCAGGCACCTCGATTAGTTCTCGAGCTTTTGGAGTACGTCGTCTTTAGGTTGGGGGGAGGGGTTTTATGCGATGGAGTTTCCCCACACTGAGTGGGTGGAGACTGAAGTTAGGCCAGCTTGGCACTTGATGTAATTCTCCTTGGAATTTGCCCTTTTTGAGTTTGGATCTTGGTTCATTCTCAAGCCTCAGACAGTGGTTCAAAGTTTTTTTCTTCCATTTCAG | 58 |
| 5′ UTR human CamKIIa | TCAGAAGCCCCGGGCTCGTCAGTCAAACCGGTTCTCTGTTTGCACTCGGCAGCACGGGCAGGCAAGTGGTCCCTAGGTTCGGG | 59 |
| B-globin intron | GTGAGTCTATGGGACCCTTGATGTTTTCTTTCCCCTTCTTTT CTATGGTTAAGTTCATGTCATAGGAAGGGGAGAAGTAACAGGGTACACATATTGACCAAATCAGGGTAATTTTGCATTTGTAATTTTAAAAAATGCTTTCTTCTTTTAATATACTTTTTTGTTTATCTTATTTCTAATACTTTCCCTAATCTCTTTCTTTCAGGGCAATAATGATACAATGTATCATGCCTCTTTGCACCATTCTAAAGAATAACAGTGATAATTTCTGGGTTAAGGCAATAGCAATATTTCTGCATATAAATATTTCTGCATATAAATTGTAACTGATGTAAGAGGTTTCATATTGCTAATAGCAGCTACAATCCAGCTACCATTCTGCTTTTATTTTATGGTTGGGATAAGGCTGGATTATTCTGAGTCCAAGCTAGGCCCTTTTGCTAATCATGTTCATACCTCTTATCTTCCTCCCACAG | 60 |
| SV40 intron | TCTAGAGGATCCGGTACTCGAGGAACTGAAAAACCAGAAAGTTAACTGGTAAGTTTAGTCTTTTTGTCTTTTATTTCAGGTCCCGGATCCGGTGGTGGTGCAAATCAAAGAACTGCTCCTCAGTGGATGTTGCCTTTACTTCTAGGCCTGTACGGAAGTGTTACTTCTGCTCTAAAAGCTGCGGAATTGTACCCGC | 61 |

In some embodiments, the vector comprises a 3′ untranslated region selected from Table 5. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 62-70.

TABLE 5

| 3′ UNTRANSLATED REGION | SEQUENCE | SEQ ID NO: |
|---|---|---|
| WPRE(x) (mutated woodchuck hepatitis regulatory element -version 1) | AATCAACCTCTGGATTACAAAATTTGTGAAAGATTGACTGGTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTATAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGGGCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGGGGCTCGGCTGTTGGGCACTGACAATTCCGTGGTGTTGTCGGGGAAATCATCGTCCTTTCCTTGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAGACGAGTCGGATCTCCCTTTGGGCCGCCTCCCCGC | 62 |
| WPRE(x) (mutated woodchuck hepatitis regulatory element -version 2) | TCAACCTCTGGATTACAAAATTTGTGAAAGATTGACTGGTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTATAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGGGCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGGGGCTCGGCTGTTGGGCACTGACAATTCCGTGGTGTTGTCGGGGAAATCATCGTCCTTTCCTTGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAGACGAGTCGGATCTCCCTTTGGGCCGCCTCCCCGCA | 63 |
| WPRE(x) (mutated woodchuck hepatitis regulatory element -version 3) | TTCCTGTTAATCAACCTCTGGATTACAAAATTTGTGAAAGATTGACTGGTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTATAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGGGCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGGGGCTCGGCTGTTGGGCACTGACAATTCCGTGGTGTTGTCGGGGAAGCTGACGTCCTTTCCGCGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCCTCTTCGCCTTCGCCCTCAGACGAGTCGGATCTCCCTTTGGGCCGCCTCCCCGCCCATGTATCTTTTTCACCTGTGCCTTGTTTTTGCCTGTGTTCCGCGTCCTACTTTTCAAGCCTCCAAGCTGTGCCTTGGGCGGCTTTGGGGCATGGACATAGATCCCTATAAAGAATTTGGTTCATCTTATCAGTTGTTGAATTTTCTTCCTTTGGAC | 64 |
| CAAX | TGTGTGATAATG | 65 |
| EES | CTGTTCTCATCACATCATATCAAGGTTATATACCATCAATATTGCCACAGATGTTACTTAGCCTTTTAATATTTCTCTAATTTAGTGTATATGCAATGATAGTTCTCTGATTTCTGAGATTGAGTTTCTCATGTGTAATGATTATTTAGAGTTTCTCTTTCATCTGTTCAAATTTTTGTCTAGTTTTATTTTTTACTGATTTGTAAGACTTCTTTTTATAATCTGCATATTACAATTCTCTTTACTGGGGTGTTGCAAATATTTTCTGTCATTCTATGGCCTGACTTTTCTTAATGGTTTTTTAATTTTAAAAATAAGTCTTAATATTCATGCAATCTAATTAACAATCTTTTCTTTGTGGTTAGGACTTTGAGTCATAAGAAATTTTTCTCTACACTGAAGTCATGATGGCATGCTTCTATATTATTTTCTAAAAGATTTAAAGTTTTGCCTTCTCCATTTAGACTTATAATTCACTGGAATTTTTTTGTGTGTATGGTATGACATATGGGTTCCCTTTTATTTTTTACATATAAATATATTTCCCTGTTTTTCTAAAAAAGAAAAAGATCATCATTTTCCCATTGTAAAATGCCATATTTTTTTCATAGGTCACTTACATATATCAATGGGTCTGTTTCTGAGCTCTACTCTATTTTATCAGCCTCACTGTCTATCCCCACACATCTCATGCTTTGCTCTAAATCTTGATATTTAGTGGAACATTCTTTCCCATTTTGTTCTACAAGAATATTTTTGTTATTGTCTTTGGGCTTTCTATATACATTTTGAAATGAGGTTGACAAGTTA | 66 |
| HPRE | ATAACAGGCCTATTGATTGGAAAGTTTGTCAACGAATTGTGGGTCTTTTGGGGTTTGCTGCCCCTTTTACGCAATGTGGATATCCTGCTTTAATGCCTTTATATGCATGTATACAAGCAAAACAGGCTTTTACTTTCTCGCCAACTTACAAGGCCTTTCTCAGTAAACAGTATATGACCCTTTACCCCGTTGCTCGGCAACGGCCTGGTCTGTGCCAAGTGTTTGCTGACGCAACCCCCACTGGTTGGGGCTTGGCCATAGGCCATCAGCGCATGCGTGGAACCTTTGTGTCTCCTCTGCCGATCCATACTGCGGAACTCCTAGCCGCTTGTTTTGCTCGCAGCAGGTCTGGAGCAAACCTCATCGGGACCGACAATTCTGTCGTACTCTCCCGCAAGTATACATCGTTTCCATGGCTGCTAGGCTGTGCTGCCAACTGGATCCTGCGCGGGACGTCCTTTGTTTACGTCCCGTCGGCGCTGAATCCCGCGGACGACCCCTCCCGGGGCCGCTTGGGGCTCTACCGCCCGCTTCTCCGTCTGCCGTACCGTCCGACCACGGGGCGCACCTCTCTTTACGCGGACTCCCCGTCTGTGCCTTCTCATCTGCCGGACCGTGTGCACTTCGCTTCACCTCTGCACGTCGCATGGAGGCCACCGTGAACGCCCACCGGAACCTGCCCAAGGTCTTGCATAAGAGGACTCTTGGACTTTCAGCAATGTCATC | 67 |
| R2V17 (HepB derived enhancer element) | TTCCTGTAAACAGGCCTATTGATTGGAAAGTTTGTCAACGAATTGTGGGTCTTTTGGGGTTTGCTGCCCCTTTTACGCAATGTGGATATCCTGCTTTAATGCCTTTATATGCATGTATACAAGCAAAACAGGCTTTTACTTTCTCGCCAACTTACAAGGCCTTTCTCAGTAAACAGTATATGACCCTTTACCCCGTTGCTCGGCAACGGCCTGGTCTGTGCCAAGTGTTTGCTGACGCAACCCCCACTGGTTGGGGCTTGGCCATAGGCCATCAGCGCATGCGTGGAACCTTTGTGTCTCCTCTGCCGATCCATACTGCGGAACTCCTAGCCGCTTGTTTTGCTCGCAGCTGGACTGGAGCAAACCTCATCGGGACCGACAATTCTGTCGTACTCTCCCGCAAGCACTCACCGTTTCCGCGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCCTCTTCGCCTTCGCCCTCAGACGAGTCGGATCTCCCTTTGGGCCGCCTCCCCGCCCATGTATCTTTTTCACCTGTGCCTTGTTTTTGCCTGTGTTCCGCGTCCTACTTTTCAAGCCTCCAAGCTGTGCCTTGGGCGGCTTTGGGGCATGGACATAGATCCCTATAAAGAATTTGGTTCATCTTATCAGTTGTTGAATTTTCTTCCTTTGGAC | 68 |
| 3 ‘UTR(globin) | GCTGGAGCCTCGGTAGCCGTTCCTCCTGCCCGCTGGGCCTCCCAACGGGCCCTCCTCCCCTCCTTGCACCGGCCCTTCCTGGTCTTTGAATAAA | 69 |
| WPRE(r) | ATTCGAGCATCTTACCGCCATTTATTCCCATATTTGTTCTGTTTTTCTTGATTTGGGTATACATTTAAATGTTAATAAAACAAAATGGTGGGGCAATCATTTACATTTTTAGGGATATGTAATTACTAGTTCAGGTGTATTGCCACAAGACAAACATGTTAAGAAACTTTCCCGTTATTTACGCTCTGTTCCTGTTAATCAACCTCTGGATTACAAAATTTGTGAAAGATTGACTGATATTCTTAACTATGTTGCTCCTTTTACGCTGTGTGGATATGCTGCTTTAATGCCTCTGTATCATGCTATTGCTTCCCGTACGGCTTTCGTTTTCTCCTCCTTGTATAAATCCTGGTTGCTGTCTCTTTATGAGGAGTTGTGGCCCGTTGTCCGTCAACGTGGCGTGGTGTGCTCTGTGTTTGCTGACGCAACCCCCACTGGCTGGGGCATTGCCACCACCTGTCAACTCCTTTCTGGGACTTTCGCTTTCCCCCTCCCGATCGCCACGGCAGAACTCATCGCCGCCTGCCTTGCCCGCTGCTGGACAGGGGCTAGGTTGCTGGGCACTGATAATTCCGTGGTGTTGTCGGGGAAGGGCC | 70 |

In some embodiments, the vector comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 71-75.

TABLE 6

| POLYADENYLATION SITE | SEQUENCE | SEQ ID NO: |
|---|---|---|
| Rabbit globin (pAGlobin-Oc) | TGGCTAATAAAGGAAATTTATTTTCATTGCAATAGTGTG TTGGAATTTTTTGTGTCTCTCACTCGGAAGAACATATGG GAGGGCAAATCATTTAAAACATCAGAATGAGTATTTGGTTTAGAGTTTGGCAACATATGCCCATATGCTGGCTGCCATGAACAAAGGTTGGCTATAAAGAGGTCATCAGTATATGAAACAGCCCCCTGCTGTCCATTCCTTATTCCATAGAAAAGCCTTGACTTGAGGTTAGATTTTTTTTATATTTTGTTTTGTGTTATTTTTTTCTTTAACATCCCTAAAATTTTCCTTACATGTTTTACTAGCCAGATTTTTCCTCCTCTCCTGACTACTCCCAGTCATAGCTGTCCCTCTTCTCTTATGGAGATC | 71 |
| Bovine growth hormone (pAGH-Bt -version 1) | TTGCCAGCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTGAGTAGGTGTCATTCTATTCTGGGGGGTGGGGTGGGGCAGGACAGCAAGGGGGAGGATTGGGAATACAATAGCAGGCATGCTGGGGATGCGGTGGGCTCTATGGGTACCCAGGTGCTGAAGAATTGACCCGGTTCCTCCTGGG | 72 |
| Bovine growth hormone (pAGH-Bt -version 2) | TTGCCAGCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTGAGTAGGTGTCATTCTATTCTGGGGGGTGGGGTGGGGCAGGACAGCAAGGGGGAGGATTGGGAAGACAATAGCAGGCATGCTGGGGATGCGGTGGGCTCTATGGGTACCCAGGTGCTGAAGAATTGACCCGGTTCCTCCTGGG | 73 |
| Bovine growth hormone (pAGH-Bt -version 3) | CTGTGCCTTCTAGTTGCCAGCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTGAGTAGGTGTCATTCTATTCTGGGGGGTGGGGTGGGGCAGGACAGCAAGGGGGAGGATTGGGAAGACAATAGCAGGCATGCTGGGGATGCGGTGGGCTCTATGG | 74 |
| Human growth hormone (pAGH-Hs) | CTGCCCGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATCATTTTGTCTGACTAGGTGTCCTTCTATAATATTATGGGGTGGAGGGGGGTGGTATGGAGCAAGGGGCCCAAGTTGGGAAGAAACCTGTAGGGCCTGC | 75 |

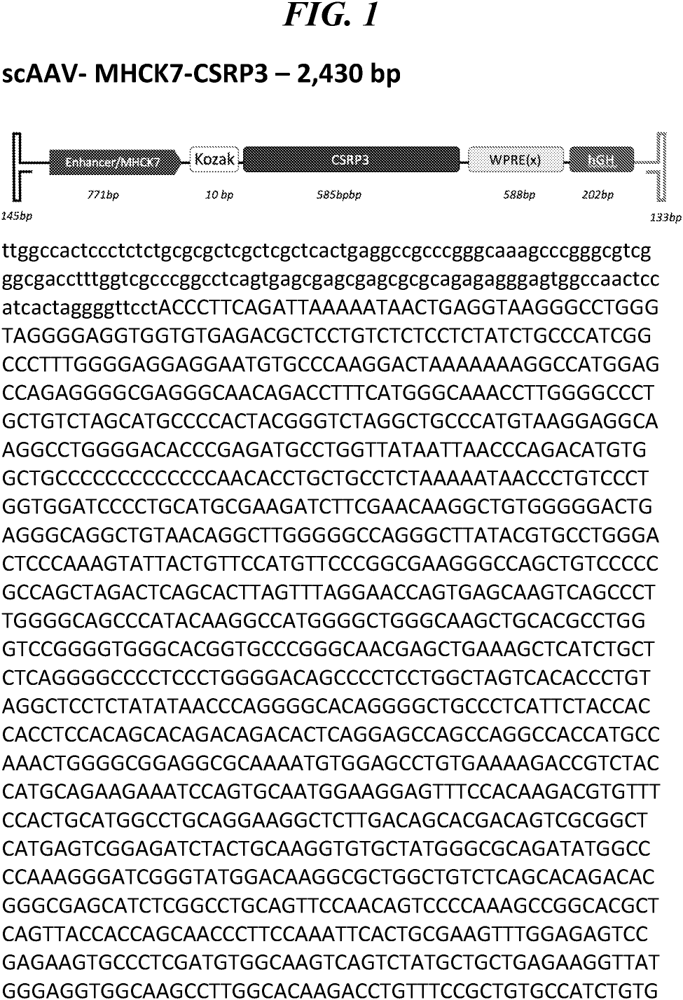

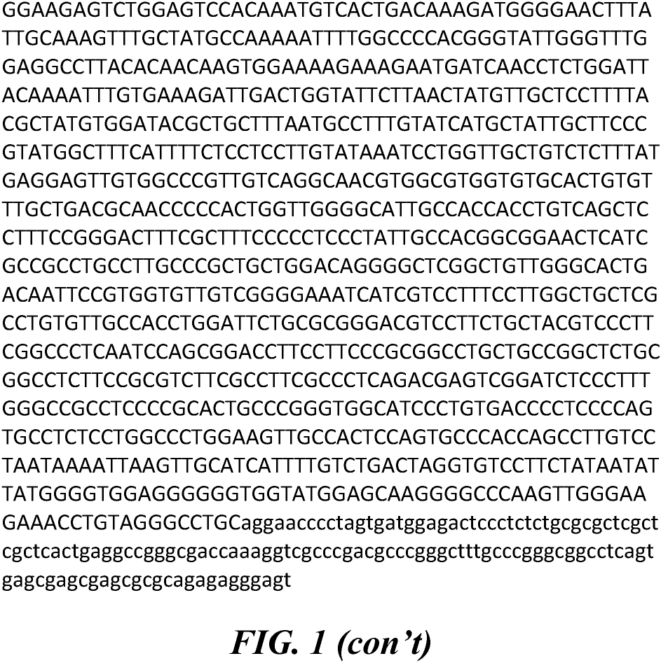

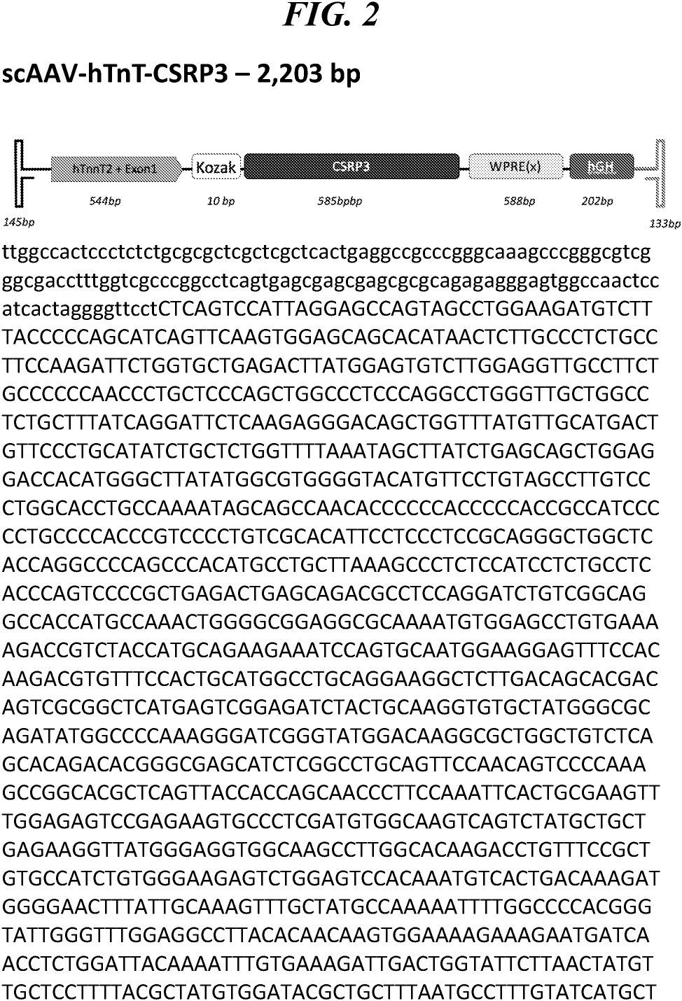

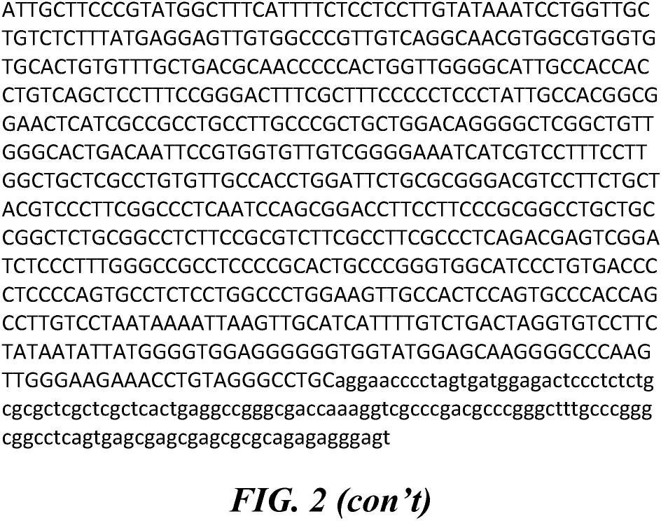

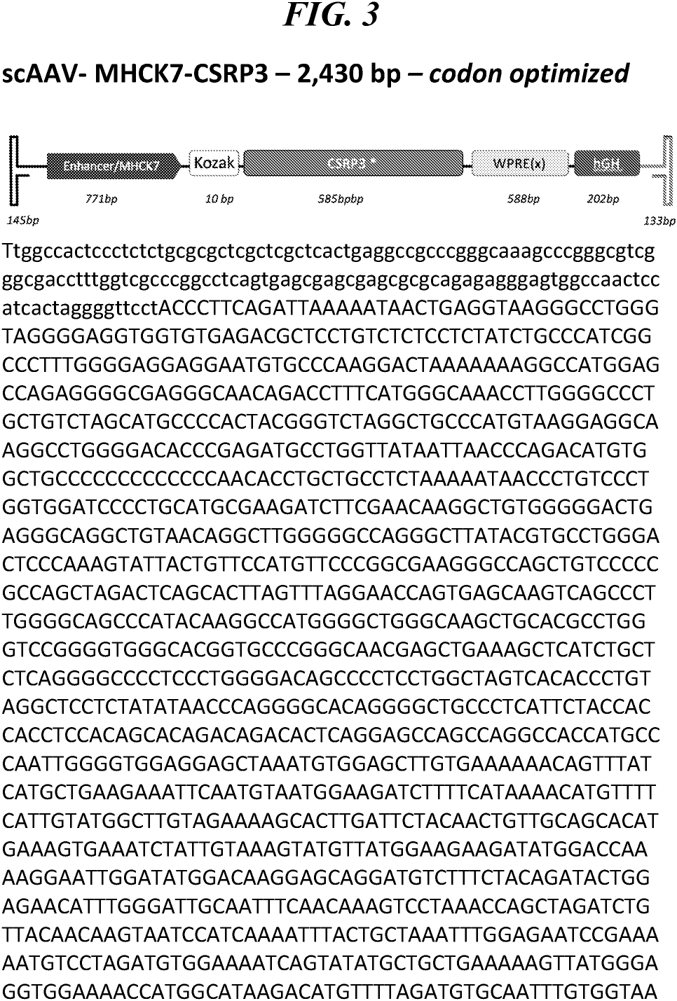

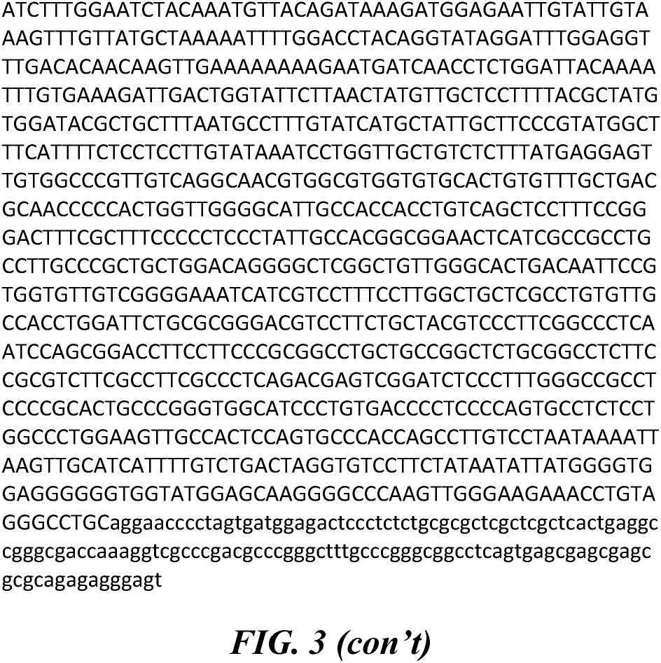

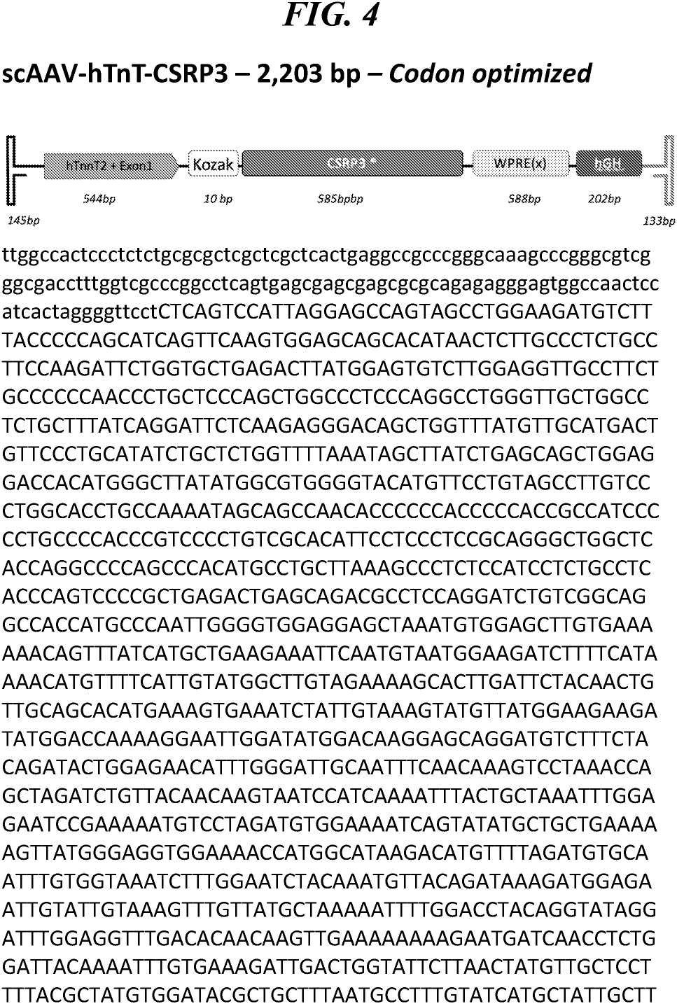

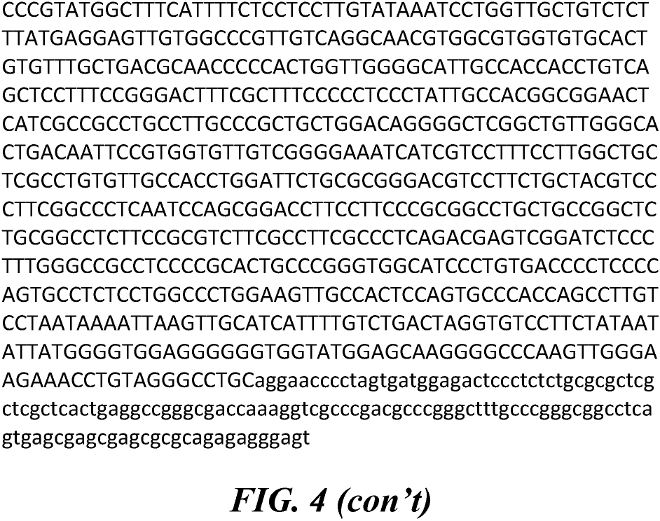

Illustrative vector genomes are depicted in FIGS. 1-4 and provided as SEQ ID NOs: 12-15. The expression cassette of each sequence, capitalized, is SEQ ID NOs: 8-11. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 8-11, optionally with or without the ITR sequences in lowercase. The coding sequence is capitalized.

### Adeno-Associated Virus Vector

Adeno-associated virus (AAV) is a replication-deficient parvovirus, the single-stranded DNA genome of which is about 4.7 kb in length including two ~145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in W095/14785, W096/22378, U.S. Pat. No. 5,882,877, U.S. Pat. No. 6,013,516, U.S. Pat. No. 4,861,719, U.S. Pat. No. 5,278,056 and W094/19478, the complete contents of each of which is hereby incorporated by reference.

AAV vectors useful in the practice of the present invention can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle DR, Russell DW. Curr Opin Mol Ther. (2009) 11(4):442-447; McCarty et al. Gene Ther. (2001) 8(16):1248-54; and Duan et al. Mol Ther. (2001) 4(4):383-91. Helper-free systems included those described in US 6,004,797; US 7,588,772; and US 7,094,604;

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV- 10, AAV-11, AAV- 12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs are have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In some cases, the rAAV vector is of the serotype AAV1, AAV2, AAV4, AAV5, AAV6, AAV7, AAV8, AAV9, AAV10, AAV11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, e.g., substitutions, deletions, or insertions, within a capsid protein, e.g., VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as endothelial cells or more particularly endothelial tip cells. In some embodiments, the rAAV is directly injected into the intracerebroventricular space of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g.,

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL(SEQ ID NO: 76)

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g.,

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL(SEQ ID NO: 77
)

In some embodiments, the rAAV virion is an AAV6 rAAV virion. The capsid many be an AAV9 capsid or functional variant thereof. In some embodiments, the AAV6 capsid shares at least 98%, 99%, or 100% identity to a reference AAV6 capsid, e.g.,

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPFGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDKDKFFPMSGVMIFGKESAGA
SNTALDNVMITDEEEIKATNPVATERFGTVAVNLQSSSTDPATGDVHVMG
ALPGMVWQDRDVYLQGPIWAKIPHTDGHFHPSPLMGGFGLKHPPPQILIK
NTPVPANPPAEFSATKFASFITQYSTGQVSVEIEWELQKENSKRWNPEVQ
YTSNYAKSANVDFTVDNNGLYTEPRPIGTRYLTRPL(SEQ ID NO: 78
)

In some embodiments, the rAAV virion is an AAVrh. 10 rAAV virion. The capsid many be an AAV9 capsid or functional variant thereof. In some embodiments, the AAVrh. 10 capsid shares at least 98%, 99%, or 100% identity to a reference AAVrh. 10 capsid, e.g.,

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGWADNLQQQNAAPIVGAVNSQ
GALPGMVWQNRDVYLQGPIWAKIPHTDGNFHPSPLMGGFGLKHPPPQILI
KNTPVPADPPTTFSQAKLASFITQYSTGQVSVEIEWELQKENSKRWNPEI
QYTSNYYKSTNVDFAVNTDGTYSEPRPIGTRYLTRNL(SEQ ID NO: 7
9)

In some embodiments, the capsid protein is encoded by a polynucleotide supplied on a plasmid in trans to the transfer plasmid. The polynucleotide sequence of wild-type AAVrh74 cap is as follows:
AAVrh74 capsid coding sequence (SEQ ID NO: 80)

ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCC
AAGCGGGTGACAATCCGTACCTGCGGTATAATCACGCCGACGCCGAGTTT
CAGGAGCGTCTGCAAGAAGATACGTCTTTTGGGGGCAACCTCGGGCGCGC
AGTCTTCCAGGCCAAAAAGCGGGTTCTCGAACCTCTGGGCCTGGTTGAAT
CGCCGGTTAAGACGGCTCCTGGAAAGAAGAGACCGGTAGAGCCATCACCC
CAGCGCTCTCCAGACTCCTCTACGGGCATCGGCAAGAAAGGCCAGCAGCC
CGCAAAAAAGAGACTCAATTTTGGGCAGACTGGCGACTCAGAGTCAGTCC
CCGACCCTCAACCAATCGGAGAACCACCAGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGCACCTGGGCCCTG
CCCACCTACAACAACCACCTCTACAAGCAAATCTCCAACGGGACCTCGGG
AGGAAGCACCAACGACAACACCTACTTCGGCTACAGCACCCCCTGGGGGT
ATTTTGACTTCAACAGATTCCACTGCCACTTTTCACCACGTGACTGGCAG
CGACTCATCAACAACAACTGGGGATTCCGGCCCAAGAGGCTCAACTTCAA
GCTCTTCAACATCCAAGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTGCTCGGCTCGGCGCACCAGGGCTGCCTGCCTCC
GTTCCCGGCGGACGTCTTCATGATTCCTCAGTACGGGTACCTGACTCTGA
ACAATGGCAGTCAGGCTGTGGGCCGGTCGTCCTTCTACTGCCTGGAGTAC
TTTCCTTCTCAAATGCTGAGAACGGGCAACAACTTTGAATTCAGCTACAA
CTTCGAGGACGTGCCCTTCCACAGCAGCTACGCGCACAGCCAGAGCCTGG
ACCGGCTGATGAACCCTCTCATCGACCAGTACTTGTACTACCTGTCCCGG
ACTCAAAGCACGGGCGGTACTGCAGGAACTCAGCAGTTGCTATTTTCTCA
GGCCGGGCCTAACAACATGTCGGCTCAGGCCAAGAACTGGCTACCCGGTC
CCTGCTACCGGCAGCAACGCGTCTCCACGACACTGTCGCAGAACAACAAC
AGCAACTTTGCCTGGACGGGTGCCACCAAGTATCATCTGAATGGCAGAGA
CTCTCTGGTGAATCCTGGCGTTGCCATGGCTACCCACAAGGACGACGAAG
AGCGATTTTTTCCATCCAGCGGAGTCTTAATGTTTGGGAAACAGGGAGCT
GGAAAAGACAACGTGGACTATAGCAGCGTGATGCTAACCAGCGAGGAAGA
AATAAAGACCACCAACCCAGTGGCCACAGAACAGTACGGCGTGGTGGCCG
ATAACCTGCAACAGCAAAACGCCGCTCCTATTGTAGGGGCCGTCAATAGT
CAAGGAGCCTTACCTGGCATGGTGTGGCAGAACCGGGACGTGTACCTGCA
GGGTCCCATCTGGGCCAAGATTCCTCATACGGACGGCAACTTTCATCCCT
CGCCGCTGATGGGAGGCTTTGGACTGAAGCATCCGCCTCCTCAGATCCTG
ATTAAAAACACACCTGTTCCCGCGGATCCTCCGACCACCTTCAATCAGGC
CAAGCTGGCTTCTTTCATCACGCAGTACAGTACCGGCCAGGTCAGCGTGG
AGATCGAGTGGGAGCTGCAGAAGGAGAACAGCAAACGCTGGAACCCAGAG
ATTCAGTACACTTCCAACTACTACAAATCTACAAATGTGGACTTTGCTGT
CAATACTGAGGGTACTTATTCCGAGCCTCGCCCCATTGGCACCCGTTACC
TCACCCGTAATCTGTAA

The disclosure further provides protein sequences for AAVrh74 VP1, VP2, and VP3, including SEQ ID NOs: 2-4, and homologs or functional variants thereof.
AAVrh74 VP1 (SEQ ID NO: 81)

MAAGGGAPMADNNEGADGVGSSSGNWHCDSTWLGDRVITTSTRTWALPTY
NNHLYKQISNGTSGGSTNDNTYFGYSTPWGYFDFNRFHCHFSPRDWQRLI
NNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPS
QMLRTGNNFEFSYNFEDVPFHSSYAHSQSLDRLMNPLIDQYLYYLSRTQS
TGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKD
NVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKN
TPVPADPPTTFNQAKLASFITQYSTGQVSVEIEWELQKENSKRWNPEIQY
TSNYYKSTNVDFAVNTEGTYSEPRPIGTRYLTRNL

AAVrh74 VP2 (SEQ ID NO: 82)

STIQVFTDSEYQLPYVLGSAHQGCLPPFPADVFMIPQYGYLTLNNGSQAV
GRSSFYCLEYFPSQMLRTGNNFEFSYNFEDVPFHSSYAHSQSLDRLMNPL
IDQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPSS
GVLMFGKQGAGKDNVDYSSVMLTSEEEIKTTNPVATEQYGVVADNLQQQN
AAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQ
KENSKRWNPEIQYTSNYYKSTNVDFAVNTEGTYSEPRPIGTRYLTRNL

AAVrh74 VP3 (SEQ ID NO: 83)

RTGNNFEFSYNFEDVPFHSSYAHSQSLDRLMNPLIDQYLYYLSRTQSTGG
TAGTQQLLFSQAGPNNMSAQAKNWLPGPCYRQQRVSTTLSQNNNSNFAWT
GATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPG
MVWQNRDVYLQGPIWAKIPHTDGNFHPSPLMGGFGLKHPPPQILIKNTPV
PADPPTTFNQAKLASFITQYSTGQVSVEIEWELQKENSKRWNPEIQYTSN
YYKSTNVDFAVNTEGTYSEPRPIGTRYLTRNL

In certain cases, the AAVrh74 capsid comprises the amino acid sequence set forth in SEQ ID NO: 2. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP1 which is set forth in SEQ ID NO: 2. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP2 which is set forth in SEQ ID NO: 3. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP3 which is set forth in SEQ ID NO: 4.

In some embodiments, the rAAV virion is an AAV-PHP.B rAAV virion or a neutrotrophic variant thereof, such as, without limitation, those disclosed in Int′l Pat. Pub. Nos. WO 2015/038958 A1 and WO 2017/100671 A1. For example, the AAV capsid may comprise at least 4 contiguous amino acids from the sequence TLAVPFK (SEQ ID NO:85) or KFPVALT (SEQ ID NO:86), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g.,

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQTLAVPFKAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL (SEQ I
D NO: 84)

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAV9 vector, AAVrh.74, or an AAVrh. 10 vector will confer desirable cardiac tropism on the vector. Without being bound by theory, the present inventors have further determined that an AAV9 vector, AAVrh.74, or an AAVrh.10 vector may provide desired specificity to cardiac cells.

### Pharmaceutical Compositions and Kits

In an aspect, the disclosure provides pharmaceutical compositions comprising the rAAV virion of the disclosure and one or more pharmaceutically acceptable carriers, diluents, or excipients.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

### Methods of Use

In an aspect, the disclosure provides a method of increasing MLP activity in a cell, comprising contacting the cell with an rAAV of the disclosure. In another aspect, the disclosure provides a method of increasing MLP activity in a subject, comprising administering to an rAAV of the disclosure. In some embodiments, the cell and/or subject is deficient in CSRP3 messenger RNA or MLP protein expression levels and/or activity and/or comprises a loss-of-function mutation in CSRP3. The cell may be a cardiac cell, e.g. a cardiomyocyte cell.

In some embodiments, the method promotes survival of cardiac cell, e.g. a cardiomyocyte cell, in cell culture and/or in vivo. In some embodiments, the method promotes and/or restores function of the heart.

### Methods of Treatment

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder. Illustrative cardiac disorders include heart failure, hypertrophic cardiomyopathy, and dilated cardiomyopathy. In some embodiments, the subject suffers from a genetic disruption in CSRP3 expression or function. In some embodiments, the disease or disorder is HCM or DCM. In some embodiments, the disease or disorder is familial hypertrophic cardiomyopathy-12 (CMH12). In some embodiments, the disease or disorder is dilated cardiomyopathy-1M (CMD1M). In some embodiments, the disease or disorder is a skeletal myopathy. In some embodiments, the disease or disorder is facioscapulohumeral muscular dystrophy, nemaline myopathy, or limb girdle muscular dystrophy type 2B. In some embodiments, the disease or disorder is limb girdle muscular dystrophy type 2A, Duchenne muscular dystrophy, or dermatomyositis.

The AAV-mediated delivery of MLP protein to the heart may increase life span, prevent or attenuate cardiac cell degeneration, heart failure, scarring, reduced ejection fraction, arrythmia, angina, obstructive HCM or DCM, ventricular hypertrophy (IVS: range 14-32 mm), ventricular tachycardia, Mild NYHA scores I-II common, exercise intolerance, angina (chest pain), sudden cardiac death, exertional myalgias and cramps.

The methods disclosed herein may provide efficient biodistribution in the heart. They may result in sustained in expression in all, or a substantial fraction of, cardiac cells, e.g., cardiomyocytes. Notably, the methods disclosed herein may provide long-lasting expression of MLP protein throughout the life of the subject following AAV vector administration.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector is administered at a dose of between about 1×10¹² and 5×10¹⁴ vector genomes (vg) of the AAV vector per kilogram (vg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵vg/kg, 5×10¹⁵ vg/kg, or 7×10¹⁵ vg/kg.

In some embodiments, the AAV vector is administered systemically at a dose of between about 1×10¹² and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered systemically at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered systemically at a dose of 1×10¹²vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg.

In some embodiments, the AAV vector is administered intravenously at a dose of between about 1×10¹² and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the AAV vector is administered intravenously at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered intravenously at a dose of 1×10¹²vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵vg/kg, 5×10¹⁵ vg/kg, or 7×10¹⁵ vg/kg.

Evidence of functional improvement, clinical benefit or efficacy in patients may be reveald by change in New York Heart Association functional classification (NYHA Class), pathological electrocardiogram, left ventricular end-diastolic/end-systolic diameter, maximal interventricular wall thickness, maximal posterior wall thickness, Peak E and Peak A Velocity, peak early and peak late transmitrial filling velocities, early diastolic and late diastolic tissue Doppler velocities, hypertension and degree of cardiac hypertrophy. Additional myocardial histology would reveal AAV-mediated MLP benefit showing reduction in hypertrophy of cardiomyocytes, reduced myocyte array and reduced interstitial and perivascular fibrosis and scaring compared to baseline or disease-matched control patients.

### Administration of Compositions

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g., a mini-osmotic pump, a depot formulation, etc. In some embodiments, the methods of the disclosure comprise administering an AAV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration.

In particular, administration of rAAV of the present invention may be accomplished by using any physical method that will transport the rAAV recombinant vector into the target tissue of an animal. Administration includes, but is not limited to, injection into the heart.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration. The compositions of the disclosure may further be administered intravenously.

The method of treatment disclosed herein may reduce and/or prevent one or more symptoms including but not limited to ventricular hypertrophy, ventricular tachycardia, mild NYHA scores I-II common, exercise intolerance, and angina.

### EXAMPLES

### Example 1: Pre-Clinical Bioactivity and Efficacy

Vectors illustrated in FIGS. 1-4 are tested in vitro using cultured cardiomyocytes (e.g., induced pluripotent stem cell cardiomyocytes, iPSC-CMs). Expression of MLP is assessed by immunofluorescence and Western blot. Phosphorylation assays reveal a reduction in protein kinase C-alpha (PKC-A) autophosphorylation.

Selected vectors are tested in vivo using an MLP-deficient or MLP-mutant mouse models of cardiomyopathy (e.g., C58G knock-in (KI) model or W4R KI model). Efficacy is determined by measuring left ventricular ejection fraction (LVEF) and/or left ventricular end-diastolic dimension (LVED) using echocardiography, reduction in overall heart weight (e.g, normalized to tibia length), invasive haemodynamic assessments of left ventricular performance on dP/dtₘₐₓ, dP/dtₘᵢₙ, and relaxation constant Tau, or reduction in left and/or right ventricular hypertrophy upon histologic evaluation. Additionally, in vivo efficacy in the mouse model is assessed by measuring biomarkers including but not limited to atrial natriuretic factor (Nppa) gene expression, brain natriuretic peptide (Nppb) gene expression, and beta-myosin heavy chain protein expression. Physiological efficacy is determined by testing for protein kinase C-alpha (PKC-A) activity, phosphorylated MLP in heart, ubiquitin proteasome degradation activity. Normalization or mitigation in response to treatment is observed for AAV vectors.

### Example 2: Protein Expression in Human Cardiomyocytes

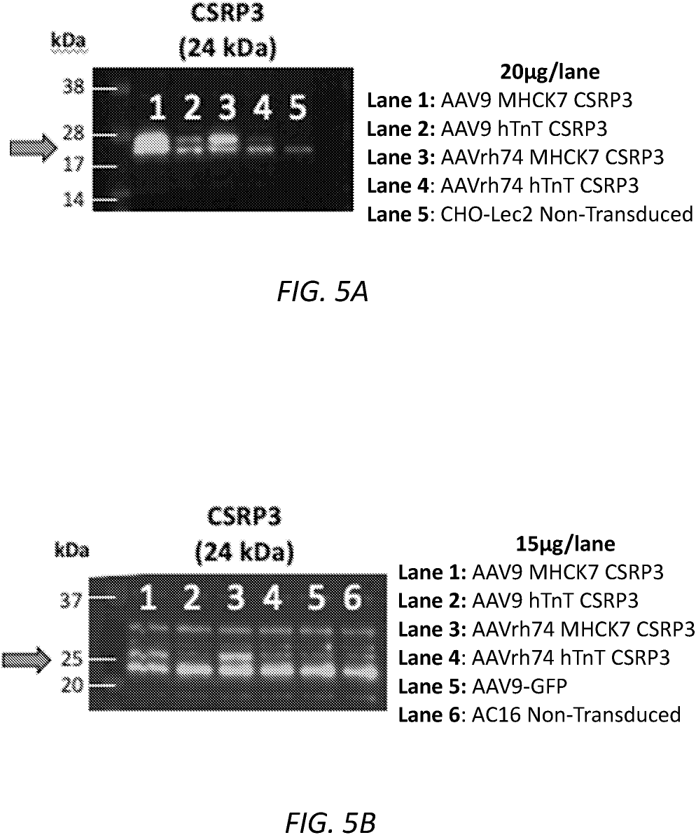

Vectors illustrated in FIGS. 1-4 were tested in vitro using a control cell line (CHO-Lec2; FIG. 5A) and cultured cardiomyocytes (differentiated AC16 cell line; FIG. 5B ). Expression of muscle LIM protein (MLP;the protein encoded by CSRP3) was assessed by Western blot.

FIG. 5A shows CSRP3 expression in transduced CHO-Lec2. FIG. 5B shows CSRP3 expression in transduced cardiomyocytes (differentiated AC16 cell line - Sigma-Aldrich® cat# SCC109). The cells were transduced with 3E5 MOI from each vector; after 6 days the cells lysates were collected, and a Western Blot performed using an anti-CSRP3 Polyclonal antibody (Thermo-Fisher® PA5-29155 1:1000).

Expression of the MLP protein from the CSRP3 transgene is higher when the MHCK7 promoter is used than when the hTNNT2 (“hTnT”) promoter is used. Both AAV9 and AAVrh74 serotypes of AAV vector are capable of transducing the cardiomyocyte cell line. Expression of the MLP protein is apparently higher with the AAVrh74 vector than with the AAV9 vector based on data in FIG. 5B.

1. A polynucleotide, comprising an expression cassette and optionally flanking adeno-associated virus (AAV) inverted terminal repeats (ITRs), wherein the polynucleotide comprises a polynucleotide sequence encoding Muscle LIM Protein (MLP) or a functional variant thereof, operatively linked to a promoter.

2. The polynucleotide of claim 1, wherein the promoter is a cardiac-specific promoter.

3. The polynucleotide of claim 1 or claim 2, wherein the promoter is a muscle-specific promoter.

4. The polynucleotide of any one of claims 1 to 3, wherein the promoter is a cardiomyocyte-specific promoter.

5. The polynucleotide of any one of claims 1 to 4, wherein the promoter is a MHCK7 promoter.

6. The polynucleotide of claim 5, wherein the MHCK7 promoter shares at least 75%, 80%, 85%, 90%, 95%, 96%, 97%, 98%, 99% or 100% identity with SEQ ID NO: 31.

7. The polynucleotide of any one of claims 1 to 4, wherein the promoter is a cardiac troponin T (hTNNT2) promoter.

8. The polynucleotide of claim 7, wherein the hTNNT2 promoter shares at least 75%, 80%, 85%, 90%, 95%, 96%, 97%, 98%, 99% or 100% identity with SEQ ID NO: 32.

9. The polynucleotide of any one of claims 1 to 8, wherein the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

10. The polynucleotide of any one of claims 1 to 4, wherein the promoter is a ubiquitous promoter, optionally a CMV promoter or a CAG promoter.

11. The polynucleotide of any one of claims 1 to 10, wherein the expression cassette comprises a poly A signal.

12. The polynucleotide of claim 11, wherein the polyA signal is a human growth hormone (hGH) polyA.

13. The polynucleotide of any one of claims 1 to 12, wherein the expression cassette comprises a Woodchuck Hepatitis Virus Posttranscriptional Regulatory Element (WPRE), optionally a WPRE(x).

14. The polynucleotide of any one of claims 1 to 13, wherein the Muscle LIM Protein (MLP) or a functional variant thereof is an MLP.

15. The polynucleotide of claim 14, wherein the MLP is a human MLP.

16. The polynucleotide of claim 14 or claim 15, wherein the MLP is MLP isoform A.

17. The polynucleotide of claim 15 or 16, wherein the MLP shares at least 75%, 80%, 85%, 90%, 95%, 96%, 97%, 98%, 99% or 100% identity with
MPNWGGGAKCGACEKTVYHAEEIQCNGRSFHKTCFHCMACRKALDSTTVA
AHESEIYCKVCYGRRYGPKGIGYGQGAGCLSTDTGEHLGLQFQQSPKPAR
SVTTSNPSKFTAKFGESEKCPRCGKSVYAAEKVMGGGKPWHKTCFRCAIC
GKSLESTNVTDKDGELYCKVCYAKNFGPTGIGFGGLTQQVEKKE(SEQ I
D NO: 1).

18. The polynucleotide of claim 14 or claim 15, wherein the MLP is MLP isoform B.

19. The polynucleotide of claim 15 or 18, wherein the MLP shares at least 75%, 80%, 85%, 90%, 95%, 96%, 97%, 98%, 99% or 100% identity with
MPNWGGGAKCGACEKTVYHAEEIQCNGRSFHKTCFHCSPQSRHAQLPPAT
LPNSLRSLESPRSALDVASQSMLLRRLWEVASLGTRPVSAVPSVGRVWSP
QMSLTKMGNFIAKFAMPKILAPRVLGLEALHNKWKRKNEEVRRFSDFLRA
(SEQ ID NO: 2).

20. The polynucleotide of claim 15, wherein the MLP shares at least 75%, 80%, 85%, 90%, 95%, 96%, 97%, 98%, 99% or 100% identity with
MPNWGGGAKCGACEKTVYHAEEIQCNGRSFHKTCFHCLC(SEQ ID NO:
3).

21. The polynucleotide of claim 15, wherein the MLP shares at least 75%, 80%, 85%, 90%, 95%, 96%, 97%, 98%, 99% or 100% identity with
MPNWGGGAKCGACEKTVYHAEEIQCNGRSFHKTCFHCTLAQDLFPLCHLW
EESGVHKC (SEQ ID NO: 4).

22. The polynucleotide of any one of claims 1 to 21, wherein the polynucleotide sequence encoding MLP is a Cysteine And Glycine Rich Protein 3 (CSRP3) polynucleotide.

23. The polynucleotide of claim 22, wherein the CSRP3 polynucleotide is a human CSRP3 polynucleotide.

24. The polynucleotide of any one of claims 1 to 23, wherein the polynucleotide sequence encoding MLP shares at least 75%, 80%, 85%, 90%, 95%, 96%, 97%, 98%, 99% or 100% identity with
ATGCCAAACTGGGGCGGAGGCGCAAAATGTGGAGCCTGTGAAAAGACCGT
CTACCATGCAGAAGAAATCCAGTGCAATGGAAGGAGTTTCCACAAGACGT
GTTTCCACTGCATGGCCTGCAGGAAGGCTCTTGACAGCACGACAGTCGCG
GCTCATGAGTCGGAGATCTACTGCAAGGTGTGCTATGGGCGCAGATATGG
CCCCAAAGGGATCGGGTATGGACAAGGCGCTGGCTGTCTCAGCACAGACA
CGGGCGAGCATCTCGGCCTGCAGTTCCAACAGTCCCCAAAGCCGGCACGC
TCAGTTACCACCAGCAACCCTTCCAAATTCACTGCGAAGTTTGGAGAGTC
CGAGAAGTGCCCTCGATGTGGCAAGTCAGTCTATGCTGCTGAGAAGGTTA
TGGGAGGTGGCAAGCCTTGGCACAAGACCTGTTTCCGCTGTGCCATCTGT
GGGAAGAGTCTGGAGTCCACAAATGTCACTGACAAAGATGGGGAACTTTA
TTGCAAAGTTTGCTATGCCAAAAATTTTGGCCCCACGGGTATTGGGTTTG
GAGGCCTTACACAACAAGTGGAAAAGAAAGAA(SEQ ID NO: 5).

25. The polynucleotide of any one of claims 1 to 24, wherein the polynucleotide sequence encoding MLP shares at least 75%, 80%, 85%, 90%, 95%, 96%, 97%, 98%, 99% or 100% identity with
ATGCCCAATTGGGGTGGAGGAGCTAAATGTGGAGCTTGTGAAAAAACAGT
TTATCATGCTGAAGAAATTCAATGTAATGGAAGATCTTTTCATAAAACAT
GTTTTCATTGTATGGCTTGTAGAAAAGCACTTGATTCTACAACTGTTGCA
GCACATGAAAGTGAAATCTATTGTAAAGTATGTTATGGAAGAAGATATGG
ACCAAAAGGAATTGGATATGGACAAGGAGCAGGATGTCTTTCTACAGATA
CTGGAGAACATTTGGGATTGCAATTTCAACAAAGTCCTAAACCAGCTAGA
TCTGTTACAACAAGTAATCCATCAAAATTTACTGCTAAATTTGGAGAATC
CGAAAAATGTCCTAGATGTGGAAAATCAGTATATGCTGCTGAAAAAGTTA
TGGGAGGTGGAAAACCATGGCATAAGACATGTTTTAGATGTGCAATTTGT
GGTAAATCTTTGGAATCTACAAATGTTACAGATAAAGATGGAGAATTGTA
TTGTAAAGTTTGTTATGCTAAAAATTTTGGACCTACAGGTATAGGATTTG
GAGGTTTGACACAACAAGTTGAAAAAAAAGAA(SEQ ID NO: 7).

26. The polynucleotide of any one of claims 1 to 25, wherein the polynucleotide comprises at least about 2.4 kb, at most about 2.6 kb, or between about 2.4 kb and about 2.6 kb.

27. The polynucleotide of any one of claims 1 to 26, wherein the polynucleotide comprises at least about 3.0 kb, at most about 3.3 kb, or between about 3.0 kb and about 3.3 kb.

28. The polynucleotide of any one of claims 1 to 27, wherein the polynucleotide comprises at least about 2.4 kb, least about 2.6 kb, least about 3.0 kb, at least about 3.3 kb, at least about 3.5 kb, at least about 3.7 kb, at least about 3.9 kb, at least about 4.1 kb., or at least about 4.3 kb.

29. The polynucleotide of any one of claims 1 to 28, wherein the polynucleotide comprises least about 2.6 kb, least about 3.0 kb, at most about 3.3 kb, at most about 3.5 kb, at most about 3.7 kb, at most about 3.9 kb, at most about 4.1 kb., at most about 4.3 kb, or at most about 4.5 kb.

30. The polynucleotide of any one of claim 1 to 29, wherein the expression cassette shares at least 75%, 80%, 85%, 90%, 95%, 96%, 97%, 98%, 99% or 100% identity with any one of SEQ ID NOs: 8-11.

31. The polynucleotide of any one of claim 1 to 30, wherein the polynucleotide shares at least 75%, 80%, 85%, 90%, 95%, 96%, 97%, 98%, 99% or 100% identity with any one of SEQ ID NOs: 12-15.

32. The polynucleotide of any one of claims 1 to 31, wherein the expression cassette is flanked by 5′ and 3′ inverted terminal repeats (ITRs), optionally AAV2 ITRs, optionally ITRs that shares at least 75%, 80%, 85%, 90%, 95%, 96%, 97%, 98%, 99% or 100% identity with any one of SEQ ID NO: 20-26.

33. The polynucleotide of any one of claim 1 to 32, wherein the polynucleotide is self-complementary.

34. The polynucleotide of any one of claim 1 to 33, wherein the polynucleotide comprises the expression cassette and a reverse complement of the expression cassette.

35. The polynucleotide of claim 34, wherein the expression cassette and the reverse complement of the expression cassette are flanked by 5′ and 3′ inverted terminal repeats (ITRs), optionally AAV2 ITRs, optionally an ITR that shares at least 75%, 80%, 85%, 90%, 95%, 96%, 97%, 98%, 99% or 100% identity with SEQ ID NO: 23 or SEQ ID NO: 26.

36. A gene therapy vector, comprising the polynucleotide of any one of claims 1 to 35.

37. The vector of claim 36, wherein the gene therapy vector is a recombinant adeno-associated virus (rAAV) vector.

38. The vector of claim 37, wherein the rAAV vector is an AAV9 or a functional variant thereof.

39. The vector of claim 38, wherein the rAAV vector comprises a capsid protein that shares 90%, 91%, 92%, 93%, 94%, 95%, 96%, 97%, 98%, 99% or 100% identity to any one of SEQ ID NO: 77.

40. The vector of claim 37, wherein the rAAV vector is an AAVrh10 or a functional variant thereof.

41. The vector of claim 40, wherein the rAAV vector comprises a capsid protein that shares 90%, 91%, 92%, 93%, 94%, 95%, 96%, 97%, 98%, 99% or 100% identity to any one of SEQ ID NO: 79.

42. The vector of claim 37, wherein the rAAV vector is an AAV6 or a functional variant thereof.

43. The vector of claim 42, wherein the rAAV vector comprises a capsid protein that shares 90%, 91%, 92%, 93%, 94%, 95%, 96%, 97%, 98%, 99% or 100% identity to any one of SEQ ID NO: 78.

44. The vector of claim 37, wherein the rAAV vector is an AAVrh74 or a functional variant thereof.

45. The vector of claim 44, wherein the rAAV vector comprises a capsid protein that shares 90%, 91%, 92%, 93%, 94%, 95%, 96%, 97%, 98%, 99% or 100% identity to any one of SEQ ID NO: 80.

46. The vector of any one of claims 36 to 45, wherein the rAAV vector is a self-complementary AAV vector.

47. A method of treating and/or preventing a disease or disorder in a subject in need thereof, comprising administering the vector of any one of claims 35 to 46 to the subject.

48. The method of claim 47, wherein the disease or disorder is a cardiac disorder.

49. The method of claim 47 or 48, wherein the disease or disorder is heart failure.

50. The method of any one of claims 47 to 49, wherein the disease or disorder is hypertrophic cardiomyopathy.

51. The method of any one of claims 47 to 49, wherein the disease or disorder is dilated cardiomyopathy.

52. The method of any one of claims 47 to 51, wherein the subject is a mammal.

53. The method of claim 52, wherein the subject is a primate.

54. The method of claim 53, wherein the subject is a human.

55. The method of any one of claims 45 to 54, wherein the subject has a mutation in the CSRP3 gene that causes an amino acid substitution selected from C58G, L44P, S54R, E55G, and/or K69R, relative to a human CSRP3 encoding a human MLP having the sequence of SEQ ID NO: 1.

56. The method of any one of claim 47 to 55, wherein the vector is administered by intravenous injection, intracardiac injection, intracardiac infusion, and/or cardiac catheterization.

57. The method of any one of claims 47 to 56, wherein the administration increases MLP expression by at least about 5%.

58. The method of any one of claims 47 to 56, wherein the administration increases MLP expression by at least about 30%.

59. The method of any one of claims 47 to 56, wherein the administration increases MLP expression by at least about 70%.

60. The method of any one of claims 47 to 56, wherein the administration increases MLP expression by about 5% to about 10%.

61. The method of any one of claims 47 to 56, wherein the administration increases MLP expression by about 30% to about 50%.

62. The method of any one of claims 47 to 56, wherein the administration increases MLP expression by about 70% to about 100%.

63. The method of any one of claims 47 to 62, wherein the method treats and/or prevents the disease or disorder.

64. A pharmaceutical composition comprising the vector of any one of claims 36 to 46.

65. A kit comprising the vector of any one of claims 34 to 46 or the pharmaceutical composition of claim 64 and optionally instructions for use.

66. Use of the composition of any one of claims 36 to 46 in treating a disease or disorder, optionally according to the method of any one of claims 47 to 63.

67. A composition according to any one of claims 36 to 46 for use in treating a disease or disorder, optionally according to the method of any one of claims 47 to 63.

68. A method of expressing Muscle LIM Protein (MLP) or a functional variant thereof, comprising contacting a cell with the vector of any one of claims 36 to 46.

69. The method of claim 68, wherein the cell is a cardiomyocyte.

70. The method of claim 69, wherein the cardiomyocyte is a human cardiomyocyte.

71. The method of any one of claims 68 to 70, wherein the promoter is an MHCK7 promoter and wherein the expression level of the MLP is at least 2-fold greater than the expression level of MLP in a cell transduced with a vector having an hTNNT2 promoter.

72. The method of any one of claims 68 to 70, wherein the promoter is an MHCK7 promoter and wherein the expression level of the MLP is between 2-fold greater and 10-fold greater than the expression level of MLP in a cell transduced with a vector having an hTNNT2 promoter.


SEQUENCE LISTING (86 sequences)

SEQ ID NO: 1 - protein, 194 aa, Homo sapiens
MPNWGGGAKCGACEKTVYHAEEIQCNGRSFHKTCFHCMACRKALDSTTVAAHESEIYCKVCYGRRYGPKGIGYGQGAGCLSTDTGEHLGLQFQQSPKPARSVTTSNPSKFTAKFGESEKCPRCGKSVYAAEKVMGGGKPWHKTCFRCAICGKSLESTNVTDKDGELYCKVCYAKNFGPTGIGFGGLTQQVEKKE

SEQ ID NO: 2 - protein, 150 aa, Homo sapiens
MPNWGGGAKCGACEKTVYHAEEIQCNGRSFHKTCFHCSPQSRHAQLPPATLPNSLRSLESPRSALDVASQSMLLRRLWEVASLGTRPVSAVPSVGRVWSPQMSLTKMGNFIAKFAMPKILAPRVLGLEALHNKWKRKNEEVRRFSDFLRA

SEQ ID NO: 3 - protein, 39 aa, Homo sapiens
MPNWGGGAKCGACEKTVYHAEEIQCNGRSFHKTCFHCLC

SEQ ID NO: 4 - protein, 58 aa, Homo sapiens
MPNWGGGAKCGACEKTVYHAEEIQCNGRSFHKTCFHCTLAQDLFPLCHLWEESGVHKC

SEQ ID NO: 5 - DNA, 582 bp, Homo sapiens
atgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaa

SEQ ID NO: 6 - DNA, 10 bp, Artificial Sequence
  Kozak sequence motif
gccaccatgg

SEQ ID NO: 7 - DNA, 588 bp, Artificial Sequence
  Polynucleotide encoding an MLP with Kozak sequence
gccaccatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaa

SEQ ID NO: 8 - DNA, 2152 bp, Artificial Sequence
  Made in Lab - MHCK7-CSRP3 expression cassette
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 9 - DNA, 1925 bp, Artificial Sequence
  Made in lab - hTnT-CSRP3 expression cassette
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 10 - DNA, 2152 bp, Artificial Sequence
  Made in Lab - MHCK7-CSRP3 expression cassette
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgcccaattggggtggaggagctaaatgtggagcttgtgaaaaaacagtttatcatgctgaagaaattcaatgtaatggaagatcttttcataaaacatgttttcattgtatggcttgtagaaaagcacttgattctacaactgttgcagcacatgaaagtgaaatctattgtaaagtatgttatggaagaagatatggaccaaaaggaattggatatggacaaggagcaggatgtctttctacagatactggagaacatttgggattgcaatttcaacaaagtcctaaaccagctagatctgttacaacaagtaatccatcaaaatttactgctaaatttggagaatccgaaaaatgtcctagatgtggaaaatcagtatatgctgctgaaaaagttatgggaggtggaaaaccatggcataagacatgttttagatgtgcaatttgtggtaaatctttggaatctacaaatgttacagataaagatggagaattgtattgtaaagtttgttatgctaaaaattttggacctacaggtataggatttggaggtttgacacaacaagttgaaaaaaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 11 - DNA, 1925 bp, Artificial Sequence
  Made in Lab - hTnT-CSRP3 express cassette codon optimized
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgcccaattggggtggaggagctaaatgtggagcttgtgaaaaaacagtttatcatgctgaagaaattcaatgtaatggaagatcttttcataaaacatgttttcattgtatggcttgtagaaaagcacttgattctacaactgttgcagcacatgaaagtgaaatctattgtaaagtatgttatggaagaagatatggaccaaaaggaattggatatggacaaggagcaggatgtctttctacagatactggagaacatttgggattgcaatttcaacaaagtcctaaaccagctagatctgttacaacaagtaatccatcaaaatttactgctaaatttggagaatccgaaaaatgtcctagatgtggaaaatcagtatatgctgctgaaaaagttatgggaggtggaaaaccatggcataagacatgttttagatgtgcaatttgtggtaaatctttggaatctacaaatgttacagataaagatggagaattgtattgtaaagtttgttatgctaaaaattttggacctacaggtataggatttggaggtttgacacaacaagttgaaaaaaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 12 - DNA, 2430 bp, Artificial Sequence
  Made in Labe - full polynucleotide sequence of vector genome
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcaggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 13 - DNA, 2203 bp, Artificial Sequence
  Made in Labe - full polynucleotide sequence of vector genome
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcaggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 14 - DNA, 2430 bp, Artificial Sequence
  Made in Labe - full polynucleotide sequence of vector genome
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatgcccaattggggtggaggagctaaatgtggagcttgtgaaaaaacagtttatcatgctgaagaaattcaatgtaatggaagatcttttcataaaacatgttttcattgtatggcttgtagaaaagcacttgattctacaactgttgcagcacatgaaagtgaaatctattgtaaagtatgttatggaagaagatatggaccaaaaggaattggatatggacaaggagcaggatgtctttctacagatactggagaacatttgggattgcaatttcaacaaagtcctaaaccagctagatctgttacaacaagtaatccatcaaaatttactgctaaatttggagaatccgaaaaatgtcctagatgtggaaaatcagtatatgctgctgaaaaagttatgggaggtggaaaaccatggcataagacatgttttagatgtgcaatttgtggtaaatctttggaatctacaaatgttacagataaagatggagaattgtattgtaaagtttgttatgctaaaaattttggacctacaggtataggatttggaggtttgacacaacaagttgaaaaaaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcaggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 15 - DNA, 2203 bp, Artificial Sequence
  Made in Labe - full polynucleotide sequence of vector genome
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgcccaattggggtggaggagctaaatgtggagcttgtgaaaaaacagtttatcatgctgaagaaattcaatgtaatggaagatcttttcataaaacatgttttcattgtatggcttgtagaaaagcacttgattctacaactgttgcagcacatgaaagtgaaatctattgtaaagtatgttatggaagaagatatggaccaaaaggaattggatatggacaaggagcaggatgtctttctacagatactggagaacatttgggattgcaatttcaacaaagtcctaaaccagctagatctgttacaacaagtaatccatcaaaatttactgctaaatttggagaatccgaaaaatgtcctagatgtggaaaatcagtatatgctgctgaaaaagttatgggaggtggaaaaccatggcataagacatgttttagatgtgcaatttgtggtaaatctttggaatctacaaatgttacagataaagatggagaattgtattgtaaagtttgttatgctaaaaattttggacctacaggtataggatttggaggtttgacacaacaagttgaaaaaaaagaatgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcaggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 16 - DNA, 13 bp, Artificial Sequence
  Kozak sequence motif
gccgccrccaugg

SEQ ID NO: 17 - DNA, 13 bp, Artificial Sequence
  Kozak sequence motif
gccgccrccaugc

SEQ ID NO: 18 - DNA, 10 bp, Artificial Sequence
  Kozak sequence motif
gacaccaugg

SEQ ID NO: 19 - DNA, 10 bp, Artificial Sequence
  Kozak sequence motif
gacaccaugc

SEQ ID NO: 20 - DNA, 141 bp, Adeno-associated virus
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 168 bp, Adeno-associated virus 2
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 22 - DNA, 170 bp, Adeno-associated virus
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 23 - DNA, 145 bp, Adeno-associated virus
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 141 bp, Adeno-associated viurs
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 168 bp, Adeno-associated virus 2
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 26 - DNA, 133 bp, Adeno-associated virus 2
aggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 27 - DNA, 124 bp, Artificial Sequence
  Made in Lab - vector filler sequence
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 28 - DNA, 84 bp, Artificial Sequence
  Made in Lab - vector filler sequence
cttctgaggcggaaagaaccagatcctctcttaaggtagcatcgagatttaaattagggataacagggtaatggcgcgggccgc

SEQ ID NO: 29 - DNA, 63 bp, Artificial Sequence
  Made in Lab - vector filler sequence
gttacccaggctggagtgcagtggcacatttctgctcactgcaacctcctcctccctgggttc

SEQ ID NO: 30 - DNA, 573 bp, Artificial Sequence
  Made in lab - CAG promoter in part Human betaherpesvirus 5
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 31 - DNA, 771 bp, Artificial Sequence
  Made in Lab - MHCK7 promoter
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 32 - DNA, 544 bp, Homo sapiens
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 33 - DNA, 502 bp, Homo sapiens
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 34 - DNA, 5464 bp, Mus musculus
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 35 - DNA, 413 bp, Gallus gallus
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 36 - DNA, 1090 bp, Homo sapiens
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 37 - DNA, 253 bp, Homo sapiens
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 38 - DNA, 281 bp, Gallus gallus
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 39 - DNA, 220 bp, Human betaherpesvirus 5
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 40 - DNA, 583 bp, Human betaherpesvirus 5
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 41 - DNA, 508 bp, Human betaherpesvirus 5
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 42 - DNA, 573 bp, Artificial Sequence; identical to SEQ ID NO: 30
  Made in lab - CAG promoter in part Human betaherpesvirus 5

SEQ ID NO: 43 - DNA, 580 bp, Artificial Sequence
  Made in lab - CAG promoter in part Human betaherpesvirus 5
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 44 - DNA, 455 bp, Homo sapiens
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 45 - DNA, 401 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 46 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 47 - DNA, 422 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 48 - DNA, 281 bp, Homo sapiens
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 49 - DNA, 851 bp, Artificial Sequence
  Made in Lab - eSYN promoter polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagg

SEQ ID NO: 50 - DNA, 304 bp, Human betaherpesvirus 5
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 51 - DNA, 953 bp, Homo sapiens
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 52 - DNA, 1068 bp, Artificial Sequence
  Made in Lab - Chicken beta-actin exon/intron plus rabbit globin intron
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 53 - DNA, 149 bp, Artificial Sequence
  Made in Lab - chimeric intron sequence
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 54 - DNA, 126 bp, Homo sapiens
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 121 bp, Human betaherpesvirus 5
tcagatcgcctggagaggccatccacgctgttttgacctccatagtggacaccgggaccgatccagcctccgcggccgggaacggtgcattggaacgcggattccccgtgccaagagtgac

SEQ ID NO: 56 - DNA, 512 bp, Artificial Sequence
  Made in Lab - adenovirus derived enhancer element
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 57 - DNA, 956 bp, Homo sapiens
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 58 - DNA, 939 bp, Homo sapiens
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 59 - DNA, 83 bp, Homo sapiens
tcagaagccccgggctcgtcagtcaaaccggttctctgtttgcactcggcagcacgggcaggcaagtggtccctaggttcggg

SEQ ID NO: 60 - DNA, 476 bp, Homo sapiens
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 196 bp, simian virus 40
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 62 - DNA, 589 bp, Artificial Sequence
  Made in Lab - mutated woodchuck hepatitis regulatory element
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, Artificial Sequence
  Made in Lab - mutated woodchuck hepatitis regulatory element
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, Artificial Sequence
  Made in Lab - mutated woodchuck hepatitis regulatory element
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, Artificial Sequence
  CAAX motif
tgtgtgataatg

SEQ ID NO: 66 - DNA, 810 bp, Homo sapiens
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, Artificial Sequence
  Made in Lab - HepB derived enhancer element
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens
gctggagcctcggtagccgttcctcctgcccgctgggcctcccaacgggccctcctcccctccttgcaccggcccttcctggtctttgaataaa

SEQ ID NO: 70 - DNA, 596 bp, Woodchuck hepatitis virus
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76 - protein, 735 aa, Adeno-associated virus 2 [sequence omitted]

SEQ ID NO: 77 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 79 - protein, 738 aa, Non-human primate Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 80 - DNA, 2217 bp, Non-human primate adeno-associated virus
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 81 - protein, 535 aa, Non-human primate adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 82 - protein, 398 aa, Non-human primate adeno-associated virus
STIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 83 - protein, 332 aa, Non-human primate adeno-associated virus
RTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 84 - protein, 743 aa, Artificial Sequence
  Synthetic construct - AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - protein, 7 aa, Artificial Sequence
  Peptide insert
TLAVPFK

SEQ ID NO: 86 - protein, 7 aa, Artificial Sequence
  Peptide insert
KFPVALT